(19) United States
(12) Patent Application Publication
(10) Pub. No.: US 2004/0034003 A1
(43) Pub. Date: Feb. 19, 2004

(54) 7-Hydroxy-16alpha-fluoro-5-androsten-17-ones and 7-hydroxy-16alpha-fluoro-5-androstan-17-ones and derivatives thereof

(76) Inventors: Arthur G. Schwartz, Perkasie, PA (US); Marvin L. Lewbart, Cherry Hill, NJ (US)

Correspondence Address: SCULLY SCOTT MURPHY & PRESSER, PC, 400 GARDEN CITY PLAZA, GARDEN CITY, NY, 11530

(21) Appl. No.: 10/427,280

(22) Filed: May 1, 2003

Related U.S. Application Data
(60) Provisional application No. 60/377,182, filed on May 1, 2002.

(51) Int. Cl. A61K 31/56

(52) U.S. Cl. 514/178

(57) ABSTRACT

The present invention relates to 7-hydroxy-16α-fluoro-5-androsten-17-one and 7-hydroxy-16α-fluoro-5-androstan-17-one derivatives and their use.

### CROSS REFERENCE TO RELATED APPLICATION

This application is claiming priority of U.S. Provisional Application No. 60/377,182 filed on May 1, 2002.

### FIELD OF THE INVENTION

The present invention relates to 7-hydroxy-16α-fluoro derivatives of androsten-17-ones and 5α-androstan-17-ones, pharmaceutical compositions containing same, and their use for treating and/or preventing cancer, hypercholesterolemia, hyperlipidemia, hypertriglyceridemia, obesity, diabetes, and auto-immune disease. In addition, these compounds are useful for retarding the aging process.

### BACKGROUND OF THE INVENTION

Dehydroepiandrosterone (DHEA) and DHEA-sulfate are major adrenal secretory products in humans. The plasma concentration of DHEA-sulfate, which next to cholesterol, is the most abundant steroid in humans, undergoes the most marked age-related decline of any known steroid.

Although DHEA-sulfate is the main precursor of placental estrogen and may be converted into active androgens in peripheral tissue, there is no obvious biological role for either DHEA or DHEA-sulfate in the normal individual. Several retrospective and prospective studies suggest that women with sub-normal levels of these steroids may be predisposed to develop breast cancer.

For example, see Brownsey, et al., “Plasma dehydroepiandrosterone sulfate levels in patients with benign and malignant breast disease,” Eur. J. Cancer, 8, 131-137 (1972); Bulbrook, et al., “Relation between urinary androgen and corticoid excretion and subsequent breast cancer,” Lancet, 2, 395-398 (1971); Rose, et al., “Plasma dehydroepiandrosterone sulfate, androstenedione and cortisol, and-urinary free cortisol excretion in breast cancer,” Eur. J. Cancer, 13, 43-47 (1977); Wang, et al., “Studies of the sulfate esters of dehydroepiandorsterone and androsterone in the blood of women with breast cancer,” Eur. J. Cancer, 10, 477-482 (1974); and Zumoff, et al., “Abnormal 24-hr mean plasma concentrations of dehydroisoandrosterone and dehydroisoandrosterone sulfate in women with primary operable breast cancer,” Cancer Research, 41, 3360-3363, September 1981.

It has also been established that DHEA is a potent non-competitive inhibitor of mammalian glucose-6-phosphate dehydrogenase (G6PDH). For example, see Oertel, et al., “The effects of steroids on glucose-6-phosphate dehydrogenase,” J. Steroid Biochem., 3, 493-496 (1972) and Marks, et al., “Inhibition of mammalian glucose-6-phosphate dehydrogenase by steroids,” Proc. Nat'l Acad. Sci, USA, 46, 477-452 (1960). Moreover, Yen, et al., “Prevention of obesity in A^{vy} mice by dehydroepiandrosterone,” Lipids, 12, 409-413 (1977), reported that long-term administration of DHEA to VY-A^{vy} mice-prevented the development of obesity without suppressing appetite.

Furthermore, it is also known that the long-term treatment of C3H mice with DHEA, in addition to reducing weight gain without suppressing appetite, markedly inhibits spontaneous breast cancer development and may delay the rate of aging. It has been observed that DHEA antagonizes the capacity of the tumor promoter, 12-0-tetradecanoylphorbol-13-acetate, to stimulate ³-thymidine incorporation in mouse epidermis and in a cultured rat kidney epithelial cell line. See, Schwartz, “Inhibition of spontaneous breast cancer formation in female C3H-A^{vy}/a mice by long-term treatment with dehydroepiandrosterone”, Cancer Res., 39, 1129-1132 (1979); and Schwartz, et al., “Dehydroepiandrosterone: an anti-obesity and anti-carcinogenic agent,” Nut. Cancer 3, 46-53 (1981).

Ben-David, et al., “Anti-hy ercholesterolemic effect of dehydroepiandrosterone in rats,” Proc. Soc. Exyt. Biol. Med., 125, 1136-1140 (1967) have observed that DHEA treatment has an anti-hypercholesterolemic effect in mice, while Coleman, et al. (Diabetes 31, 830, 1982) report that administration of DHEA produces a marked hypoglycemic effect in C57BL/KsJ-db/db mice. The latter authors suggest that the therapeutic effect of DHEA might result from its metabolism to estrogens.

It is further known that DHEA and 16α-bromo-epiandrosterone are inhibitors of Epstein-Barr virus-induced transformation of human lymphocytes and that 16α-bromo-epiandrosterone is a more potent inhibitor of mammalian G6PDH than DHEA. See, Schwartz, et al. Carcinogensis, Vol. 2 No. 7, 683-686 (1981).

While DHEA has been found effective in the afore-described manners, there is however, evidence of estrogenic and androgenic effects after prolonged administration. DHEA is not an estrogen per se but is well known to be convertible into estrogens. In addition, the therapeutic dose of DHEA is rather high. It would therefore be highly desirable to provide steroids, which while having the same afore-described advantage of DHEA are more potent and do not produce an estrogenic effect.

Besides DHEA, other steroids are known in the art.

Great Britain Patent No. 989,503 to Burn, et al. discloses 6,16β-dimethyl3β-hydroxyandrost-5-en-17 -ones. These compounds are disclosed to be useful as possessing pituitary inhibiting action.

U.S. Pat. No. 2,833,793 to Dodson, et al. discloses 1β,3β-dihydroxy-5androsten-17-one as an androgenic and anabolic agent.

U.S. Pat. No. 2,911,418 to Johns, et al. discloses 16α-chloro-3β-phydroxyandrost-5-en-17-one and 3β-hydroxy-16α-iodoandrost-5-en-17-one as an anti-androgen.

Goldkamp, et al. in U.S. Pat. No. 3,148,198 disclose that 16α,16β-difluoro-3β-hydroxyandrost-5-en-17-one possess androgenic properties.

French Application No. FR-A 2,317,934 discloses the following compounds:

3β-hydroxy-16ε-methylandrost-5-en-17-one

3β-hydroxy-16ε-ethylandrostat-5-en-17-one

3β-hydroxy-16ε-isopropylandrost-5-en-17-one

U.S. Pat. No. 3,976,691 discloses the following compounds:

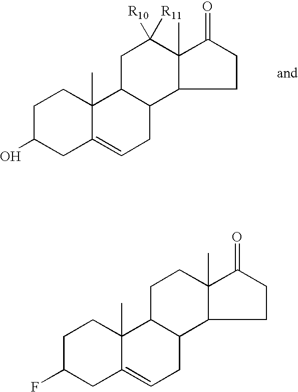

U.S. Pat. No. 3,471,480 to Fritsch, et al. discloses the following compounds which are useful as progestational agents:

(a) 3β-iodo-Δ⁵-6-methyl-17-oxoandrostene,

(b) 3β-chloro-Δ⁵-6-methyl-17-oxoandrostene, and

(c) 3β-hydroxy-Δ⁵-6-methyl-17-oxoandrostene

Hanson, et al. in Perkin Transactions I, 1977, pp. 499-501, disclose 3β,4β-dihydroxyandrost-5-en-17-one. No utility is disclosed.

Chemical Abstract 89:105866b discloses that 3β-hydroxy-5α-androstan-17one can be hydroxylated in the 15α-position. Furthermore, said reference teaches that hydroxylation of 3β-hydroxy-5α androsten-17-one gave both the 7α and 7β-hydroxyisoandrosterones.

Numazawa, et al. in Steroids, 32, 519-527 disclose 3β,16α-dihydroxyandrost-5-en-17-one. No utility is disclosed.

DE-A-2, 035,738 discloses 7α-Methyl-3β-hydroxy-5-androsten-17-one and 6,7α-dimethyl-3β-hydroxy-5-androsten-17-one.

DE-A2 705917 discloses 3α,16β-dihydroxy-5-androsten-17-one.

The Annual Report of the Fels Research Institute, pp. 32-33, (1979-1980) discloses the following compounds as having tumor-preventive, anti-obesity and anti-aging qualities:

3β-hydroxy-16α-bromo-5α-androstan-17-one,

3β-hydroxy-16α-chloro-5α-androstan-17-one,

3β-hydroxy-16α-fluoro-5α-androstan-17-one,

3β-hydroxy-16α-iodo-5α-androstan-17-one,

3β-hydroxy-16α-bromoandrost-5-en-17-one, and

16α bromoandrostan-17-one

Abou-Gharbia, et al. in Journal of Pharmaceutical Sciences, 70, 1154-1156 (1981) disclose the syntheses of:

3β-hydroxy-16α-chloro-5α-androstan-17-one,

3β-hydroxy-16α-fluoro-5α-androstan-17-one,

3β-hydroxy-16α-bromo-5α-androstan-17-one,

3β-hydroxy-16α-iodo-5α-androstan-17-one.

Pashko, et al. in Carcinogenesis, 2, 717-721 (1981) disclose that 16α-Br-epiandrosterone is more active than DHEA in inhibiting G6PDH and in reducing the rate of [³H] thymidine incorporation into mouse breast epithilum and epidermis. The authors suggest that this compound may be useful in suppressing breast cancer development.

Neef, et al. in J. Org. Chem, 43, 4679-4680 (1978) disclose the syntheses of 3β-hydroxy-16α-methyl-5-androsten-17-one and 3β-hydroxy-16β-methyl-5-androsten-17-one.

Robinson, et al. in Journal of Org. Chem., 28, 975-980 (1963) disclose the synthesis of 3β-hydroxy-16α,16β-difluoro-5-androsten-17-one.

Raineri, et al. in Biochemistry, 9, 2233-2243 (1970) tested the inhibitory activity of the following steroids on NADP and NAD linked activity of glucose 6-phosphate dehydrogenase:

3β-hydroxy-5α-androstan-17-one,

3β-hydroxy-5-androstan-17-one,

3α-hydroxy-5α-androstan-17-one,

11β-hydroxy-5α-androstan-17-one,

3α-hydroxy-4α-methyl-5α androstan-17-one,

3α-hydroxy-7α-methyl-5α androstan-17-one,

3β-hydroxy-7α-methyl-5β androstan-17-one,

3β-hydroxy-16α-bromo-5α androstan-17-one, and

3β-chloro-5α-androstan-17-one.

U.S. Pat. Nos. 5,804,576 and 5,714,481 describe 5-androsten-17-ones having the formula:

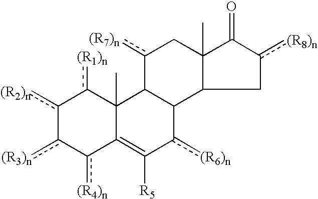

wherein R₁, R₂, R₃, R₄, R₆, R₇ and R₈ are each independently selected from the group consisting of hydrogen, alkyl, alkenyl, alkynyl, halogen and hydroxyl; Rₛ is hydrogen, alkyl, alkenyl, alkynyl or halogen, n is an integer from 1 to 2 inclusive with the proviso that when R₁, R₂, R₃, R₄, R₅, R₆, R₇ or R₈ is alkenyl or alkynyl, n is 1; and with the further provisos that at least one of R₁, R₂, R₃, R₄, R₅, R₆, R₇ or R₈ is other than hydrogen; that when R₃ is hydroxy, any one of the substituents R₂, R₄, R, R₆, R₇ or R₈ is other than hydrogen and R₁ is other than hydrogen or hydroxy; when R₃ is hydroxy, R₁ may only be alkyl when any one of R₂, R₄, R₅, R₆, R₇ or R₈ is other than hydrogen; when R₃ is hydroxy, R₄ may only be halogen or hydroxy when R₁, R₂, R₅, R₆, R₇ or R₈ is other than hydrogen; when R₃ is hydroxy, R₆ may only be hydroxy when R₁, R₂, R₄, R₅, R₇ or R₈ is other than hydrogen; when R₃ is hydroxy, R₂ may only be alkyl when one of R₁, R₄, R₅, R₆, R₇ or R₈ is other than hydrogen; when R₃ is hydroxy, R₆ can only be methyl when R₁, R₂, R₄, R₇ or R₈ is other than hydrogen and R₅ is other than hydrogen or methyl; when R₃ is hydroxy, R₇ may only be hydroxy when R₁, R₂, R₄, R₅, R₆ or R₈ is other than hydrogen; when R₃ is hydroxy, R₈ may only be methyl, ethyl, isopropyl, hydroxy or halogen when R₁, R₂, R₄, R₅, R₆ or R₇ is other than hydrogen; when R₃ is hydroxy, R₅ may only be alkyl when R₁, R₂, R₄ or R₇ is other than hydrogen and R₆ or R₈ is other than hydrogen or methyl; when R₃ is fluorine, any one of the substituents R₁, R₂, R₄, R₅, R₆, R₇ or R₈ is other than hydrogen; when R₃ is iodine or chlorine, R₅ may only be methyl when R₁, R₂, R₄, R₆, R₇ or R₈ is other than hydrogen; and when R₃ is hydroxy, R₄ may only be hydroxy when R₁, R₂, R₅, R₆ or R₈ is other than hydrogen.

They also disclose 16α-fluoro-5α-androstan-17-ones of the formula:

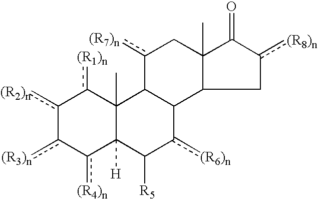

wherein R₁, R₂; R₃, R₄, R₆, R₇ or R₈ are selected from the group consisting of hydrogen, alkyl, alkenyl, alkynyl, halogen and hydroxyl, R₅ is hydrogen, alkyl, alkenyl, hydroxy, alkynyl or halogen, n is an integer from 1 to 2 inclusive with the proviso that when R₁-R₈ are alkenyl or alkynyl, then n is 1 and with the further provisos that R₃ may be hydroxy or halogen only when any one of R₁, R₂, R₄, R₅, R₆, R₇ or R₈ is other than hydrogen; when R₃ is hydroxy, R₁ may be hydroxy or halogen only when any one of R₂, R₄, R₅, R₆, R₇ or R₈ is other than hydrogen; when R₃ is hydroxy, R₂ may be methyl or halogen only when any one of R₄, R₅, R₆, R₇ or R₈ is other than hydrogen; when R₃ is hydroxy, R₄ may be halogen, methyl or hydroxy only when any one of R₁, R₂, R₃, R₅, R₆, R₇ or R₈ is other than hydrogen; when R₃ is hydroxy, R₅ may be methyl, halogen or hydroxy only when R₁, R₂, R₄, R₇ or R₈ is other than hydrogen; when R₃ is hydroxy, R₆ may be hydroxy or methyl only when R₁, R₂, R₄, R₅, R₇ or R₈ is other than hydrogen; when R₃ is hydroxy, R₇ may be hydroxy only when R₁, R₂, R₄, R₅, R₆ or R₈ is other than hydrogen; when R₃ is hydroxy, R₈ may be methyl, hydroxy or halogen only when R₁, R₂, R₄, R₅, R₆ or R₇ is other than hydrogen; R₇ may be only hydroxy when anyone of R₁, R₂, R₃, R₄, R₅, R₆ and R₈ is other than hydrogen; and R₈ may be bromo only when R₁, R₂, R₃, R₄, R₅, R₆ or R₇ is other than hydrogen.

They describe that these compounds are useful for treating, inter alia obesity, diabetes and hyperlipidemia, and for the prophylaxis of cancer.

U.S. Pat. Nos. 5,744,462, 5,700,793, 5,696,106, 5,656,621, and 5,157,031 describe steroids of the formula:

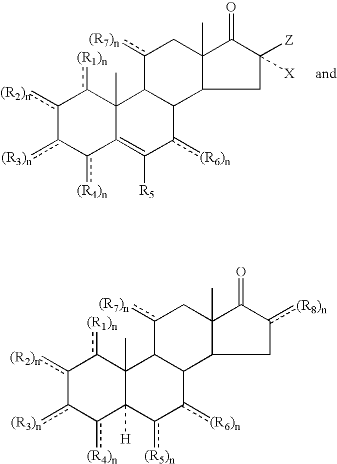

wherein

R₁, R₂, R₄, R₅, R₆ and R₇ are each independently hydrogen or lower alkyl;

R₃ is hydrogen;

X is halogen, hydroxy, hydrogen, lower alkyl, or lower alkoxy;

Z is lower alkyl or hydrogen; and

n is 1 or 2, with the proviso that at least one of X and Z is other than hydrogen.

They teach that these compounds are useful for treating various diseases.

U.S. Pat. No. 5,001,119 disclose compounds of the formula:

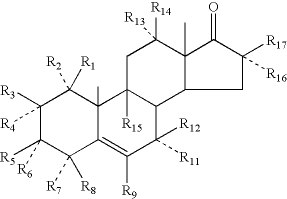

wherein

R₁, R₂, R₃, R₄, R₅, R₆, R₇, R₈, R₁₁, R₁₂, R₁₃, R₁₄ and R₁₅ are undependent hydrogen, lower alkyl, halogen, hydroxy or lower alkoxy;

R₉ is hydrogen, lower alkyl or halogen; and

R₁₆ and R₁₇ are independently hydrogen, amino, loweralkylamino, diloweralkylamino, aminoloweralkyl, loweralkyl aminolower alkyl, diloweralkylaminolower alkyl, loweralkoxyloweralkyl, lower alkoxy, hydroxy lower alkyl, monohaloloweralkyl, dihaloloweralkyl, trihaloloweralkyl, loweralkanoyl, formyl, lower carbalkoxy, or lower alkanoyloxy or R₁₆ and R₁₇ taken together with the carbons to which they are attached form a lower cycloalkyl or a cyclic ether containing one ring oxygen atom and up to 5 ring carbon atoms with the proviso that when R₅ is hydroxy and R₁, R₂, R₃, R₄, R₆, R₇, R₈, R₉, R₁₁, R₁₂, R₁₃, R₁₄ and R₁₅ are hydrogen, then R₁₆ is other than CH₂N(CH₃) and with the further proviso that R₁₆ and R₁₇ are not hydrogen simultaneously.

It also discloses compounds of the formula:

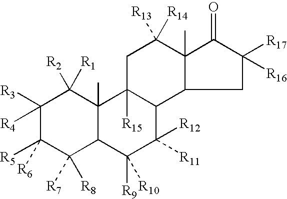

wherein

R₁, R₂, R₃, R₄, R₅, R₆, R₇, R₈, R₁₁, R₁₂, R₁₃, R₁₄ and R₁₅ are independently hydrogen, lower alkyl, halogen, hydroxy or lower alkoxy;

R₉ and R₁₀ are independently loweralkyl, hydrogen or halogen; and

R₁₆ and R₁₇ are independently amino, lower alkylamino, diloweralkyl amino, aminoloweralkyl, loweralkyl aminoloweralkyl, diloweralkylamino loweralkyl, lower alkoxy, hydroxyloweralkyl, monohaloloweralkyl, dihaloloweralkyl, trihaloloweralkyl, loweralkoxyloweralkyl, loweralkanoyl, formyl, lower carbalkoxy, hydrogen or lower alkanoyloxy; or

R₁₆ and R₁₇ taken together with the carbon to which they are attached form a lower cycloalkyl or a cyclic ether containing one ring oxygen atom and up to 5 ring carbon atoms, with the further proviso that R₁₆ and R₁₇ are not hydrogen simultaneously.

It discloses that these compounds are useful pharmaceuticals.

However, the present inventors have found that 7-hydroxy-16α-fluoro-5androsten-17-ones and 7-hydroxy-16α-fluoro-5α-androstan-17-ones and derivatives thereof have advantages and properties not heretofore known and appreciated, which makes them extremely effective as a drug.

### SUMMARY OF THE INVENTION

Accordingly the present invention is directed to 7-hydroxy-16α-fluoro compounds of the formula

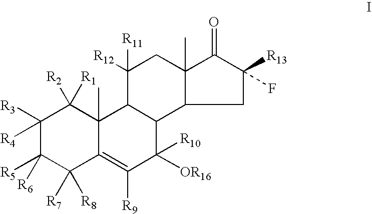

wherein

R₁, R₂, R₃, R₄, R₇, R₈, R₉, R₁₀, R₁₁, R₁₂, R₁₃ and R₁₆ and independently hydrogen or alkyl,

R₅ and R₆ are independently hydrogen or alkyl.

The present invention s also directed to compounds of the forrnula

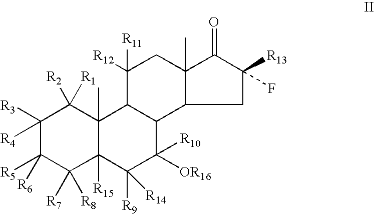

wherein

R₁, R₂, R₃, R₄, R₅, R₆, R₇, R₈, R₉, R₁₀, R₁₁ and R₁₂ and R₁₃ and R₁₄ and R₁₆ are independently hydrogen or alkyl; and

R₁₅ is hydrogen, hydroxy or alkyl.

The compounds hereinabove are useful for treating cancer and/or for the prophylaxis of cancer.

In addition, the compounds herein are useful as anti-obesity agents.

Further, the compounds hereinabove are anti-hypercholesterolemic agents.

Moreover, the compounds herein are anti-hyperglycemic agents. In addition, the compounds herein are useful anti-aging agents, i.e., they retard the aging process.

The compounds of the present invention are useful for the treatment and/or prevention of cancer, obesity, aging, diabetes, and/or hyperlipidemia, including hypercholesterolemia and hypertriglyceridemia.

Thus, the present invention is also directed to a method for treating a disease, malady, illness or condition selected from the group consisting of cancer, obesity, diabetes and hyperlipidemia in a mammal which process comprises administering to said mammal a therapeutically effective amount of the above-identified compounds.

The present invention is also directed to a method for the prophylaxis of cancer, obesity, aging, diabetes, and/or hyperlipidemia in a mammal which comprises administering a prophylatically effective amount of a compound of Formula I and II.

The present invention is also directed to a method of preventing and/or treating hypercholesterolemia and hypertriglyceridemia in a mammal which comprises administering to said mammal a prophylatically and/or therapeutically effective amount, respectively, of the above-identified compounds.

### DETAILED DESCRIPTION OF THE INVENTION

The compounds described herein are steroids. In accordance with IUPAC nomenclature, the carbon atoms on the steroid rings are numbered as followed:

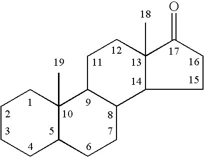

In the compounds depicted, the carbons at positions 1, 2, 3, 4, 6, 7, 11 and 16 of the steroidal ring may be unsubstituted or substituted. The other positions of the steroidal ring are not substituted, i.e., they are substituted by hydrogen. For example, in the compounds of the present invention, the 15-position of the steroidal ring is bonded to two hydrogen atoms. Except for the carbon atom at position 6 of the 5-androstenderivatives, the carbon atoms at positions 1, 2, 3, 4, 7, 11 and 16 may be unsubstituted, monosubstittued or disubstituted. The carbon atom at position 6 of the androstene derivatives are either unsubstituted or monosubstiuted. On the other hand, the carbon atom at position 6 as well as at positions 1, 2, 3, 7, 11 and 16 of the 5-androstane derivatives are either unsubstituted, monosubstituted or disubstituted. If substituted, the substitutents at position 6 of the androstane derivatives and at positions 1, 2, 3, 7, 11 and 16 of both the androstane and androstene derivatives may be in the α or β positions. If stereochemistry is to be designated, the alpha position will be designated by means of a broken line (---------) joining the substituent to the steroid nucleus and by a triangle (Δ) designating the β position. If no stereochemistry is intended, the substituent will be drawn as ˜˜˜˜ or a straight line (—).

As used herein, the term “alkyl”, when used alone or in combination has 1-12 carbon atoms. The term “lower alkyl”, refers to an alkyl group having one to six carbon atoms. The alkyl groups may be straight chain or branched. Examples include methyl, ethyl, propyl, isopropyl, butyl, isobutyl, sec-butyl, t-butyl, pentyl, neopentyl, and hexyl. It is preferred that the alkyl group is lower alkyl. The preferred lower alkyl group contains 1-3 carbon atoms. The most preferred alkyl group is methyl.

The term “alkoxy” when used alone or in combination as used herein, refers to an alkoxy group having 1-12 carbon atoms. As used herein, the term “lower alkoxy” refers to an alkoxy group having 1-6 carbon atoms. It may be straight chain or branched. Examples include methoxy, ethoxy, propoxy, isopropoxy, butoxy, isobutoxy, pentoxy and the like. It is more preferred that alkoxy contains 1-3 carbon atoms. The most preferred alkoxy group is methoxy.

The halo atoms are preferably Br, I and especially Cl and most especially F.

The fluoro substituent which is depicted hereinabove in the 16-position is in the α-position.

The OR₁₆ group at carbon 7 of the steroidal ring may be in the α-or β-position. It is preferred that the OR₁₆ group is in the β-position.

The preferred groups for R₁, R₂, R₃, R₄, R₇, R₈, R₉, R₁₀, R₁₁, R₁₂, R₁₃ and R₁₄ are hydrogen or lower alkyl, especially methyl. The most preferred group is hydrogen.

It is more preferred that there are no more than four of R₁, R₂, R₃, R₄, R₇, R₈, R₉, R₁₁, R₁₂, R₁₃ in the androstene and no more than four of R₁, R₂, R₃, R₄, R₇, R₈, R₉, R₁₀, R₁₁, R₁₂ and R₁₄ in the case of androstanes are other than hydrogen and more preferably that no more than two or three of the substituents are other than hydrogen. It is more preferred that no more than one of these groups is other than hydrogen. It is most preferred that all are hydrogen.

In addition, it is preferred that either one or both of R₁ and R₂ are hydrogen, that either one or both of R₃ and R₄ are hydrogen and that either one or both of R₇ and R₈ are hydrogen, and that either one or both of R₁₁, and R₁₂ are hydrogen.

It is also preferred that R₁₃ is hydrogen or methyl, especially hydrogen.

It is preferred that R₁₀ is hydrogen or methyl, but especially hydrogen.

It is preferred that R₁₃ is hydrogen or methyl, but especially methyl.

It is preferred that OR₁₆ is hydroxy or methoxy but especially hydroxy.

OR₁₆ may be in the alpha or beta position. Although both positions are contemplated within the present invention it is preferred that OR₁₆ is in the β-position.

It is preferred that R₅ and R₆ are independently hydrogen or lower alkyl, e.g., alkyl of 1-3 carbons. It is preferred that R₆ is in the beta position and R₅ is the α-position and that R₆, when present in the β-position, is methyl or hydrogen and that R₅ is hydrogen. It is most preferred that R₅ and R₆ are both hydrogen.

R₁₅ is preferably hydroxy, or methoxy but especially hydrogen.

It is preferred that the compounds of Formula I have the formula IA and IB.

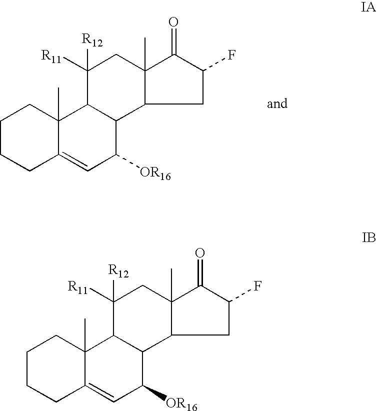

wherein R₁₁ an d R₁₂ and R₁₆ are as defined hereinabove. Compounds of Formula IB are preferred. It is more preferred that R₁₁ and R₁₂ are hydrogen. It is even more preferred that R₁₆ is hydrogen. It is especially preferred that R₁₁, R₁₂ and R₁₆ are hydrogen.;

It is also preferred that the compounds of Formula II have the Formula IIA and IIB

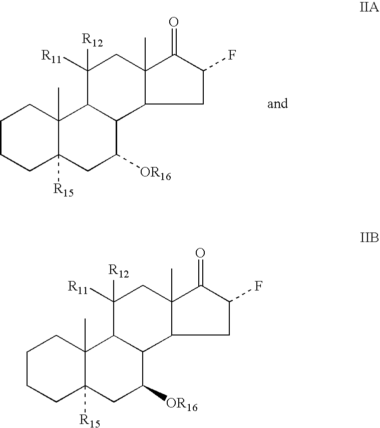

wherein R₁₁ and R₁₂, R₁₅ and R₁₆ are as defined hereinabove. Compounds of Formula IIB are preferred. It is preferred that R₁₅ is hydrogen, methoxy or hydroxy. It is more preferred that R₁₁ and R₁₂ are hydrogen. It is even more preferred that R₁₆ is hydrogen. It is even more preferred that R₁₅ is hydroxy and especially hydrogen.

It is to be understood that in the formula depicted hereinabove, the various combinations and permutations of the various definitions of R₁-R₁₆ are contemplated to be within the scope of the compounds utilized in the present invention.

The most preferred embodiments are

16α-fluoro-7β-hydroxy-5-androsten-17-one,

16α-fluoro-7α-hydroxy-5-androsten-17-one,

16α-fluoro-7β-hydroxy-5α-androstan-17-one, and

16α-fluoro-7α-hydroxy-5α-androstan-17-one.

Compounds of Formula I and II are prepared using art recognized chemical syntheses.

An exemplary procedure for the preparation of 16α-fluoro-7-hydroxy-5androsten-17-ones of the present invention is illustrated below:

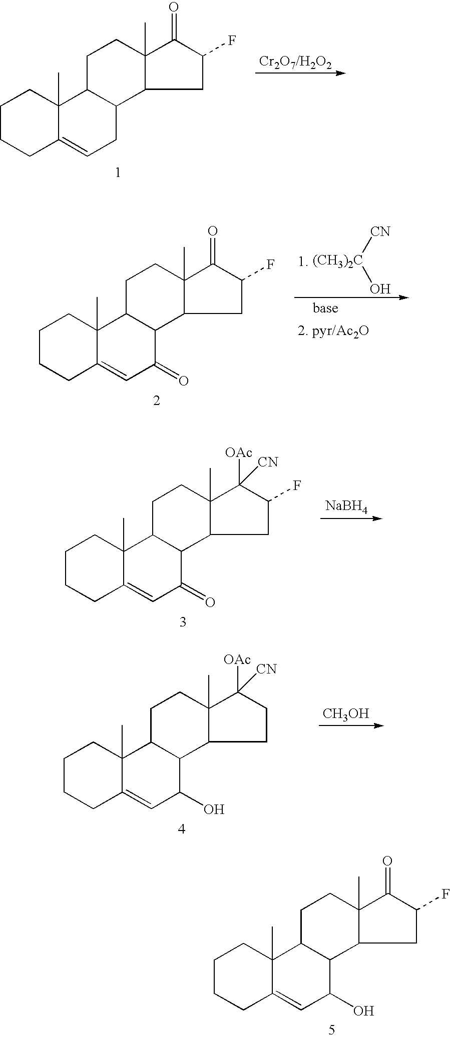

Allylic oxidation of 16-α-fluoro-5-androsten-17-one (1) with a strong oxidizing agent, such as dichromate and organic peroxides, for example, pyridinium dichromate and t-butyl hydroperoxide, provides the 16α-fluoro-5-androsten-7, 17-dione (2). The keto group at the 17-position was protected using protecting groups known in the art. For example, 2 is reacted with acetone cyanohydrin in a base, such as triethyl amine to form the 17-cyanohydrin which is then reacted with an organic acid especially C₁-C₁₆ organic acid or a derivative thereof (ester, anhydride) in the presence of base, such as acetic anhydride in pyridine to form the compound (3). The 7-keto group of 3 is reduced with a reducing agent such as sodium borohydride to formn the 7-OH compound (4). Removal of the 17-keto protecting group from 4 such as by reacting (4) with hydroxide in alcohol affords a mixture of 16α-and 16β-fluoro 7-hydroxy-5androsten-17-one which can be separated using separation techniques known in the art, such as chromatography e.g., high performance liquid chromatography on a silica gel column using as an eluent, hexane-ethyl acetate in a linear gradient 0-40% ethyl acetate to afford the 16α-fluoro-7α-hydroxy-5-androsten-17-one-and the 16α-fluoro-7β-hydroxy-5-androstan-17-one.

The procedures for making 16α-fluoro-5-androstene-17-one is described in U.S. Pat. No. 5,700,713, the contents of which is incorporated by reference.

An exemplary procedure for preparing the 5,7-dihydroxy-5-androstan-17-one is as follows, illustrated using 16α-fluoro-5-androsten-17-one.

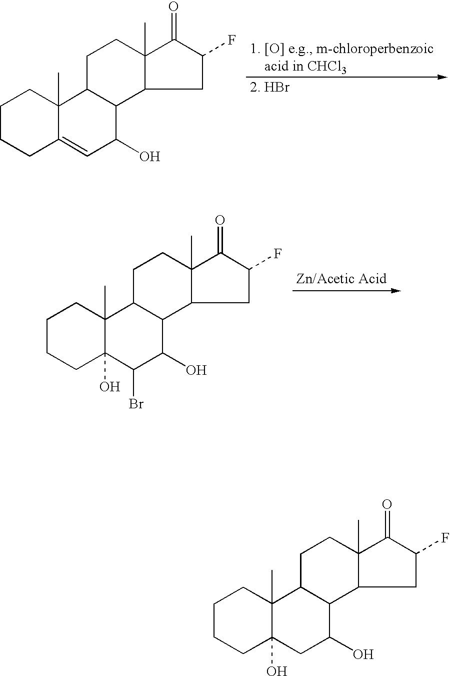

Oxidation of the double bond at the 5,6 position of the androstene using oxidizing agents known in the art to form epoxides such as m-chloroperbenzoic acid under Prilezhaev reaction conditions affords the corresponding epoxide at the 5,6 position. Reaction thereof with aqueous acid, such as HBr affords the bromohydrin. The bromide is removed by reacting the bromohydrin with zinc and acetic acid to form the 5,7-dihydroxy-16α-fluoro-5-androstan-17-one.

An exemplary procedure for the preparation of 16α-fluoro-7-hydroxy-5α-androstan-17-ones is illustrated below:

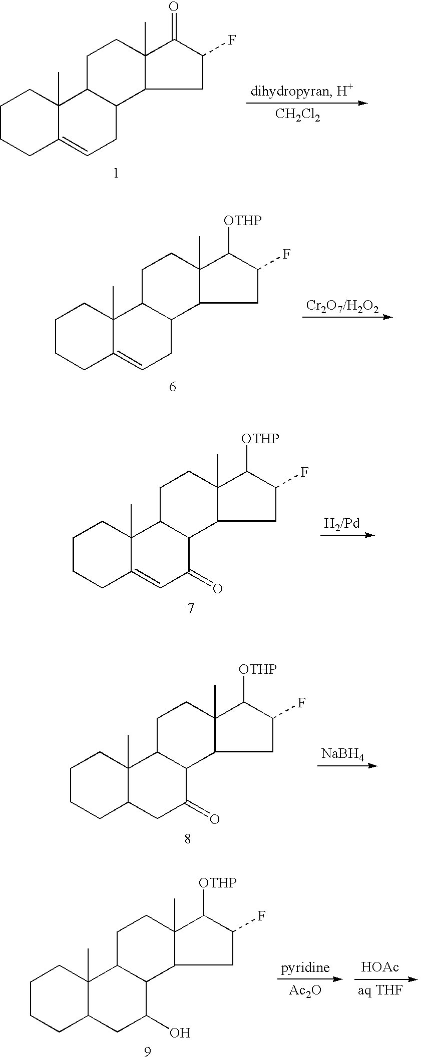

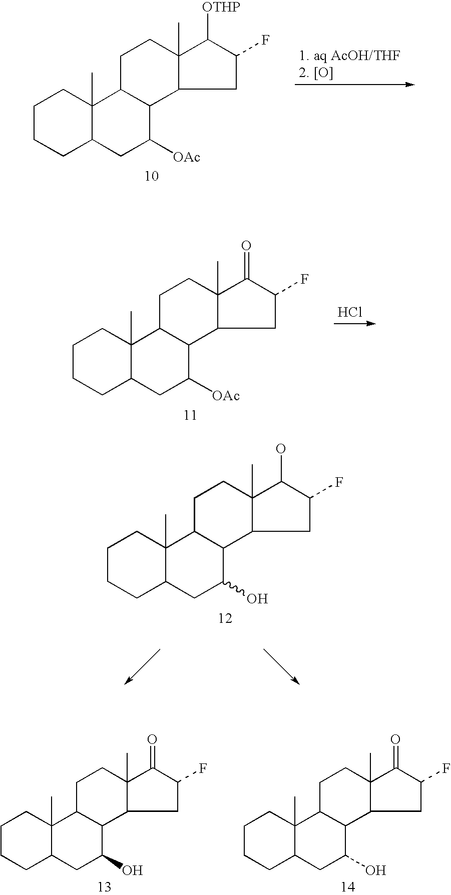

The 17-keto group is protected by reacting 16α-fluoro-5-androsten-17-one (1) with a protecting group known in the art that will protect it from reduction with reducing agents, for example, dihydropyran in the presence of acid, such as TsOH or HCl, HBr, HNO₂ and the like to afford a 17-keto-protected derivative such as 17-OTHP. derivative (6). The resulting product is subjected to allylic oxidation using standard oxidizing agents known in the art, such as chromate salts, e.g., sodium chromate, pyridinium chromate, and the like in combination with an organic peroxide e.g., lower alkyl peroxide, e.g., t-butyl hydroperoxide, to provide preferentially following flash chromatography the 7-keto-17-keto protected derivative (7), e.g., as illustrated, 7-keto-17-THP derivative. The 5,6 carbon carbon double bond is reduced with a reducing agent known in the art, such as by hydrogenation to produce the 7-keto-17-protected androstane, 17-keto-17-THP derivative (8). 8 is reacted with a reducing agent that reduces carbonyls, such as sodium borohydride to afford the corresponding 7-hydroxy androstane derivative (9). The 7-hydroxy group is protected using protecting groups in the art that protects alcohols from oxidation. For example, 9 may be reacted with pyridine/acetic anhydride, forming the 7-protected acetate derivative (10). Following removal of the protecting group at C-17 with reagents known in the art, e.g., removal of THP by subjecting 9 to aqueous acetic acid/THF, oxidation of 10 with an oxidizing agent, such as dichromate under Jones oxidation reaction conditions affords the 17-keto derivatives. The 7-hydroxy protecting group is removed by techniques known in the art. For example, in the case of acetate, the 7-acetoxy group is removed by acid hydrolysis. The resulting product from the removal of the hydroxy protecting group is 12, which is a mixture of both 7α and 7β hydroxy derivatives. The 7α and 7β derivatives can be separated using separation techniques known in the art such as chromatography, e.g., HPLC to afford the 16α-fluoro-7α-hydroxy-5α-androstan-17-one (14) and 16α-fluoro-7α-hydroxy-5α-androstan-17-one (13).

These procedures indicated hereinabove are exemplary; however, the chemistry therein is applicable in preparing the compounds of Formula I and II herein.

The alkoxy groups at the various positions denoted i.e., the 5 and 7 positions in compounds I and II are derived from the corresponding alcohols and are prepared by art recognized techniques. The methoxy substituent for example is formed by reacting the corresponding alcohol in methylene chloride with boron trifluoride and etheral diazomethane according to the procedure of Caserio, et al., JACS, 80, 2584 (1958). Similarly, the ethoxy substituent is formed by reacting the corresponding alcohol in methylene chloride with boron trifluoride and etheral diazoethane, generated in situ. Alternatively, the alkoxy substituents can also be added to the steroid ring by reacting the alcohol under Williamson reaction conditions with RX in the presence of a base, where X is an organic leaving group such as halide tosylate or mesylate and R is loweralkyl. Any base normally employed to deprotonate an alcohol may be used, such as sodium hydride, sodium amide, sodium, sodium hydroxide, triethylamino or disopropyl ethylarrine. Reaction temperatures are in the range of −78° C. to reflux. The reaction is carried out in a solvent that will dissolve both reactants and is inert to both reactants and products as well. Solvents include, but are not limited to, diethyl ether, tetrahydrofuran, N,N-dimethylformamide, methylene chloride, and the like.

The ketone should be protected with protecting groups known in the art. Examples of many of the possible protecting groups that may be utilized are found in “Protective Groups in Organic Synthesis,” by T. W. Green, John Wiley and Sons, 1981. For example, the ketone may be protected as the ethyleneketal.

The other substituents on the carbon atoms at the other positions of the steroid can be added to the steroidal ring using techniques known in the art. Exemplary procedures for these substituents are described in U.S. Pat. Nos. 5,804,576, 5,744,462, 5,714,481, 5,700,793, 5,696,106, 5,656,621, 5,157,031 and 5,001,119, the contents of all of which are incorporated by reference. If substituents on the steroidal ring are themselves reactive under the reaction conditions, then these substituents can themselves be protected utilizing protecting groups according to chemical techniques known in the art. A variety of protecting groups known in the art may be employed. Examples of many of these possible groups can be found in “Protective Groups in Organic Synthesis,” by J. W. Green, John Wiley and Sons, 1981.

If more than one substituent is to be added to the steroidal ring, the substituents can be added in any order except that it is preferred that the halogens are added last.

Finally, it should be noted that the procedures described in the aforementioned patents are applicable to all of the steroids contemplated to be utilized in the present invention. Moreover, the steroids of Formula II can be prepared from the corresponding steroids of Formula I by techniques known to one skilled in the art, e.g., by catalytic hydrogenation using, e.g., H₂/Pd, H₂/Pt or H₂/Ni, provided that the 7-hydroxy group is first protected by a protecting group that will not react under reducing conditions, for example, converting the hydroxy group at the 7-position to the t-butyl ether, diphenylsilyl ether and the like and then removing the protecting groups after the reduction of the carbon carbon double bond at the 5, 6 position by techniques known to one of ordinary skill in the art.

The compounds utilized in the present method are used in therapeutically effective amounts for therapy or prophylactically effective amount for prevention.

The physician will determine the dosage of the present therapeutic agents which will be most suitable and it will vary with the form of administration and the particular compound chosen, and furthermore, it will vary depending upon various factors, including but not limited to the patient under treatment and the age of the patient, the severity of the condition being treated and the like. He will generally wish to initiate treatment with small dosages substantially less than the optimum dose of the compound and increase the dosage by small increments until the optimum effect under the circumstances is reached. It will generally be found that when the composition is administered orally, larger quantities of the active agent will be required to produce the same effect as a smaller quantity given parenterally. The compounds are useful in the same manner as comparable therapeutic agents and the dosage level is of the same order of magnitude as is generally employed with these other therapeutic agents. When given parenterally, the compounds are administered generally in dosages of, for example, about 0.1 to about 100 mg/kg/day, also depending upon the host and the severity of the condition being treated and the compound utilized.

In a preferred embodiment, the compounds utilized are orally administered in amounts ranging from about 4 mg to about 35 mg per kilogram of body weight per day, depending upon the particular mammalian host and more preferably from about 6 to about 28 mg/kg body weight per day. This dosage regimen may be adjusted by the physician to provide the optimum therapeutic response. For example, several divided doses may be administered daily or the dose may be proportionally reduced as indicated by the exigencies of the therapeutic situation.

The compounds of Formulae I or II may be administered in a convenient manner, such as by oral, intravenous, intramuscular or subcutaneous or buccal routes.

The compounds of Formula I or II may be administered, for example, with an inert diluent or with an assimilable edible carrier, or it may be enclosed in hard or soft shell gelatin capsules, or it may be compressed into tablets, or it may be incorporated directly into the food of the diet. For therapeutic administration, the compounds of Formula I or II may be incorporated with excipients and used in the form of ingestible tablets, buccal tablets, troches, capsules, elixirs, suspensions, syrups, wafers, and the like. Such compositions and preparations should contain at least 1% of active compound of Formula I or II. The percentage of the compositions and preparations may, of course, be varied and may conveniently be between about 5 to about 80% of the weight of the unit. The amount of the compound of Formula I or II used in such therapeutical compositions is such that a suitable dosage will be obtained. Preferred compositions or preparations according to the present invention contain between about 200 mg and about 4000 mg of active compound of Formula I or II.

The tablets, troches, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier.

Various other materials may be present as coatings or otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active compound may be incorporated into sustained-release preparations and formulations. For example, sustained release dosage forms are contemplated wherein the active ingredient is bound to an ion exchange resin which, optionally, can be coated with a diffusion barrier coating to modify the release properties of the resin or wherein the active ingredient, i.e., a compound of Formula I or II, is associated with a sustained release polymer known in the art, such as hydroxypropylmethylcellulose and the like.

The active compound may also be administered parenterally or intraperitoneally. It is especially advantageous to formulate parenteral compositions in dosage unit form for ease of administration and uniformity of dosage. Dispersions can also be prepared in glycerol, liquid polyethylene glycols, e.g., PEG 100, PEG 200, PEG 300, PEG 400, and the like, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersions. In all cases the form is usually sterile and must be fluid to the extent that syringability exists. It must be stable under the conditions of manufacture and storage and usually must be preserved against the contaminating action of microorganisms such as bacteria and fungi. The carrier can be a solvent or dispersion medium containing, for example, water, ethanol, polyol (for example, glycerol, propylene glycol, and one or more liquid polyethylene glycol, e.g. as disclosed herein and the like), suitable mixtures thereof, and vegetable oils. The proper fluidity can be maintained, for example, by the use of a coating such as lecithin, by the maintenance of the required particle size in the case of dispersions and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders, the above solutions are vacuum dried or freeze-dried, as necessary.

The compounds of Formula I or Formula II can also be applied topically, as e.g., through a patch using techniques known to one of ordinary skill in the art.

The active ingredients, that is, compounds of Formula I and/or II can be administered buccally by preparing a suitable formulation of the compounds of the present invention and utilizing procedures well known to those skilled in the art. These formulations are prepared with suitable non-toxic pharmaceutically acceptable ingredients. These ingredients are known to those skilled in the preparation of buccal dosage forms. Some of these ingredients can be found in Remington's Pharmaceutical Sciences, 17^{th} edition, 1985, a standard reference in the field. The choice of suitable carriers is highly dependent upon the exact nature of the buccal dosage form desired, e.g., tablets, lozenges, gels, patches and the like. All of these buccal dosage forms are contemplated to be within the scope of the present invention and they are formulated in a conventional manner. Preferably, an effective amount of active ingredient in the buccal form ranges from about 0.15 mg/Kg to 1.5 mg/Kg.

For example, the buccal dosage form comprises the compound of Formula I or II in therapeutically effective amounts, as defined herein in association with a pharmaceutically acceptable polymer carrier, preferably a biodegradable polymer that adheres to the wet surface of the buccal mucosa and which is biodegradable and which is described in more detail hereinbelow. In one embodiment, the buccal dosage form comprises the compounds of Formula I or II in effective amounts and the polymer. However, other excipients may optionally be present, e.g., binders, disintergrants, lubricants, diluents, flavorings, colorings, and the like.

Ideally, the carrier comprises a polymer having sufficient tack to ensure that the dosage unit adheres to the buccal mucosa for the necessary time period, i.e., the time period during which the compounds of Formula I and II are to be delivered to the buccal mucosa. Additionally, it is preferred that the polymeric carrier is gradually bioerodible, i.e., the polymer hydrolyzes at a predetermined rate upon contact with moisture. The polymeric carrier is preferably sticky when moist, but not when dry, for convenience in handling. Generally, it is preferred that the average molecular weight of the polymer range from about 4,000 to about 1,000,000 g. One of skill in the art will appreciate that the higher the molecular weight of the polymer the slower the erosion time.

Any polymeric carrier can be used that is pharmaceutically acceptable, provides both a suitable degree of adhesion and the desired drug release profile and is compatible with the agents to be administered and any other components that may be present in the buccal dosage unit. Generally, the polymeric carriers comprise hydrophilic (water-soluble and water-swellable) polymers that adhere to the wet surface of buccal mucosa. Examples of polymeric carrier useful herein include acrylic acid polymers and copolymers, e.g., those known as “carbomers” (Carbopol™ which may be obtained from GAF); vinyl polymers and copolymers; polyvinyl pyrrolidone, dextran, guar gum, pectins, starches; and cellulose polymers, such as hydroxypropyl methylcellulose (e.g., Methocelo® obtainable fromQ Dow Chemical Company), hydroxypropyl cellulose (e.g., Klucel™, which may also be obtained from Dow), hydroxypropyl cellulose ethers (see, e.g., U.S. Pat. No. 4,704,285 to Alderman), hydroxyethyl cellulose, sodium carboxymethyl cellulose, methyl cellulose, ethyl cellulose, cellulose acetate phthalate, cellulose acetate butyrate, and the like. The carrier may also comprise two or more suitable polymers in combination, for example, a carbomer combined in approximately 1:5 to 5:1 ratio, by weight, with a polyethylene oxide.

The present dosage unit comprises the active agent and the polymeric carrier. However, it may be desirable in some cases to include one or more additional components. For example, a lubricant may be included to facilitate the process of manufacturing the dosage units; lubricants may also optimize erosion rate and drug flux. If a lubricant is present, it will present therein on the order of 0.01 wt. % to about 2 wt. %, preferably about 0.01 wt. % to 0.5 wt, %, of the dosage unit. Suitable lubricants include, but are not limited to, magnesium stearate, calcium stearate, steanc acid, sodium stearylfumarate, talc, hydrogenated vegetable oils and polyethylene glycol. As will be appreciated by those skilled in the art, however, modulating the particle size of the components in the dosage unit and/or the density of the unit can provide a similar effect—i.e., improved manufacturability and optimization of erosion rate and drug flux—without addition of a lubricant.

Other components may also optionally be incorporated into the buccal dosage unit. Such additional optional components include, for example, one or more disintegrants, diluents, binders, enhancers, or the like. Examples of disintegrants that may be used include, but are not limited to, cross linked polyvinylpyrrolidones, such as crospovidone (e.g., Polyplasdone® XL, which may be obtained from GAF), cross-linked carboxylic methylcelluloses, such as croscanmelose (e.g., Ac-di-sol®, which may be obtained from FMC), alginic acid, and sodium carboxymethyl starches (e.g., Explotab®, which may be obtained from Edward Medell Co., Inc.), agar bentonite and alginic acid. Suitable diluents are those which are generally useful in pharmaceutical formulations prepared using compression techniques, e.g., dicalcium phosphate dihydrate (e.g., Di-Tab®, which may be obtained from Stauffer), sugars that have been processed by crystallization with dextrin (e.g., co-crystallized sucrose and dextrin such as Di-Pak(®, which may be obtained from Amstar), lactone, calcium phosphate, cellulose, kaolin, mannitol, sodium chloride, dry starch, powdered sugar and the like. Binders, if used, are those that enhance adhesion. Examples of such binders include, but are not limited to, starch, gelatin and sugars such as sucrose, dextrose, molasses, and lactose. Permeation enhancers may also be present in the novel dosage units in order to increase the rate at which the active agents pass through the buccal mucosa. Examples of permeation enhancers include, but are not limited to, dimethylsulfoxide (“DMSO”), dimethylformamide (“DMF”), N,N-dimethylacetamide (“DMA”), decylmethylsulfoxide (“C₁₀MSO”), polyethylene glycol monolaurate (“PEGML”), glycerol monolaurate, lecithin, the 1-substituted azacycloheptan-2-ones, particularly 1-ndodecylcyclazacycloheptan-2-one (available under the trademark Azone® from Nelson Research & Development Co., Irvine, Calif.), lower alkanols (e.g., ethanol), SEPA® (available from Macrochem Co., Lexington, Mass.), cholic acid, taurocholic acid, bile salt type enhancers, and surfactants such as Tergitol®, Nonoxynol-9(® and TWEEN-80®.

Flavorings may be optionally included in the buccal formation. Any suitable flavoring may be used, e.g., mannitol, lactose or artificial sweeteners such as aspartame. Coloring agents may be added, although again, such agents are not required. Examples of coloring agents include any of the water-soluble FD&C dyes, mixtures of the same, or their corresponding lakes.

In addition, if desired, the present dosage units may be formulated with one or more preservatives or bacteriostatic agents, e.g., methyl hydroxybenzoate, propyl hydroxybenzoate, chlorocresol, benzalkonium chloride, or the like.

In general, the dosage unit of the invention is compositionally a substantially homogeneous, substantially uniform formulation. By “substantially uniform” is meant that the dosage unit is not coated, does not have a backing and does not contain a plurality of layers or other types of discrete segments. Rather, the substance of the dosage unit is similar throughout, so that the unit is essentially “monolithic” in nature.

The buccal dosage units may be in the form of tablets made by either conventional compression or molding methods. See, e.g., Remington's Pharmaceutical Sciences, 18th edition (Easton, Pa.: Mack Publishing Co., 1990). Preferably, the dosage units are prepared by mixing the components together and compressing the mixture into tablet form. As will be appreciated by those skilled in the art, the erosion rate of the dosage unit, and thus the rate of drug delivery, is controlled by three factors: the pressure used to make the tablets, and thus the tablets' density; the carrier selected, as alluded to above; and the carrier-to-drug ratio. Pressure, carrier and carrier-to-drug ratio may thus be varied to obtain shorter acting or longer-lived dosage units.

The buccal dosage units may have any of the conventional shapes, for example, lozenges, disks, wafers, tablets or the like.

The dosage unit should have dimensions which fit conveniently into the buccal cavity. By way of example, suitable dimensions for the dosage unit are 2 mm to about 5 mm in diameter, preferably not exceeding about 5 mm in diameter, and about 0.3 to about 2 mm in thickness, preferably about 0.5 to 1.5 mm in thickness, most preferably about 0.5 to 1.1 mm in thickness. The total weight of the dosage unit may be from about 5 mg to about 20 mg, preferably 10 mg to about 15 mg.

The buccal dosage units may also be generated by a molding process. Preferably, the final unit should have a melting point which is high enough to prevent fusion of packaged dosage units during shipping and storage, yet low enough to permit mixing of pharmaceutical ingredients without significant decomposition of the active agents when being incorporated into the molten carrier.

The most preferred mode of administration is the buccal form. The preferred buccal form is a tablet, and more preferably, a tablet containing fluasterone. In a preferred embodiment, the buccal tablet comprises by weight 16% fluasterone, 72%. mannitol, 7% crospovidone, 2% magnesium stearate, 1% polyethylene glycol, e.g. PEG 3350, 1% sodium lauryl sulfate and 1% amorphous silica dioxide. The buccal form has advantages over the oral form. Without wishing to be bound, it is believed that the buccal form of administration avoids the disadvantages encountered with oral drug administration, e.g., degradation of the steroid by fluids present in the gastrointestinal tract and/or first-pass inactivation in the liver and/or intestines. Moreover, unlike the oral form, the administration of the drug buccally enhances the efficacy thereof relative to oral administration. Further, it decreases the androgenicity of the drug, as compared to the oral mode of administration. This is important, especially since increased androgenicity counteracts the anti-diabetic effect of the drug. In addition, oral administration of steroids tends to lower HDL (high density lipoproteins) in men and women, an undesirable side effect. However, when the compounds of Formula I and II are administered buccally, these androgenic side effects, such as HDL lowering observed when the drug is given in high concentration during oral therapy, is significantly reduced, if not eliminated.

As used herein, “pharmaceutically acceptable carrier” includes any and all solvents, dispersion media, coatings, antibacterial and antifungal agents, isotonic and absorption delaying agents for pharmaceutical active substances well known in the art. Except insofar as any conventional media or agent is incompatible with the active ingredient, their use in the therapeutic compositions is contemplated. Supplementary active ingredients can also be incorporated into the compositions.

Dosage unit form as used herein refers to physically discrete units suited as unitary dosages for the subjects to be treated; each unit containing a predetermined quantity of active material calculated to produce the desired therapeutic effect in association with the required pharmaceutical carrier.

The principal active ingredient which is a compound of Formula I or II is compounded for convenient and effective administration in effective amounts with a suitable pharmaceutically acceptable carrier in dosage unit form as hereinbefore described. A unit dosage, for example, contains the principal active compound in amounts ranging from about 10 mg e.g. in humans, or as low as 1 mg (for small animals) to about 2000 mg. If placed in solution, the concentration of the compounds of Formula I or Formula II preferably ranges from about 10 mg/mL to about 250 mg/mL. In the case of compositions containing supplementary active ingredients, the dosages are determined by reference to the usual dose and manner of administration of the said ingredients. In the case of buccal administration, the compounds of Formula I or II are preferably in the buccal unit dosage form present in an amount ranging from about 10 to about 50 mg.

As used herein, the term “patient” or “subject” refers to a warm blooded animal and preferably mammals such as for example, cats, dogs, horses, cows, pigs, mice, rats, monkeys, apes, and humans. The preferred patient is humans.

The present invention provides methods for the prophylaxis of cancer, obesity, aging, diabetes, hyperlipidemia, and auto-immune diseases such as lupus erythematosis, Coomb's positive hemolytic anemia, multiple sclerosis, and the like comprising administering to a host, e.g. mammals a prophylactically effective amount of the compounds of Formula I or II herein.

As defined herein, with respect to the prophylaxis of aging, the compounds contemplated for use in the present invention retard the aging process by slowing down the reactions that are involved in aging.

Moreover, the present invention provides methods for the treatment of cancer, obesity, diabetes, hyperlipidemia and autoimmune disease, which comprises administering to a host, e.g., mammals a therapeutically effective amount of a compound of Formula I and II.

As used herein, the term “treat” refers to management and care of a mammalian subject, preferably human, for the purpose of combating the disease, condition or disorder and includes the administration of a compound of the present invention to either (a) prevent or delay the onset of the disease, malady or illness or the symptoms or complications associated therewith (b) to combat the disease, malady, illness and medically improve the condition, disease, malady or illness which the host is afflicted with or alleviate the symptoms or complications associated therewith or (c) eliminate the disease, condition or disorder.

The term “prophylaxis” refers to the prevention or a measurable reduction in likelihood of a mammalian subject, preferably human acquiring a disease or malady condition. If a patient is suffering from a disease, malady or disease, the term also refers to the prevention of the disease becoming exacerbated.

Unlike DHEA, the compounds of Formula I and II do not exhibit an estrogen or androgen effect.

Moreover, the compounds of Formula I and II are inhibitors of purified bovine adrenal G6PDH. This activity is one predictor of its cancer preventive action. The assay for testing the inhibition of purified bovine adrenal GGPDH is described in an article by Oertel, G. W. and Rebeleen I., in Biochem. Biophys, Acta 184, 459-460 (1969).

The compounds of the present invention also inhibit the tumor promoter stimulation of mouse epidermal DNA synthesis. This is also another indication of its cancer preventive activity. The compounds of the present invention are useful for inhibiting DMBA produced papillomas and carcinomas.

The compounds of the present invention are useful for treating various cancers, especially solid tumors. It prevents and/or retards the spread of cancer once administered thereto. Examples of the type of cancers which the compounds of the present inventor are useful in treating and/or preventing include skin cancer, lung cancer, breast cancer, colon cancer, prostate cancer, and the like.

Moreover, the administration of compounds I and II to hosts have an anti-obesity effect, that is, the hosts lose weight. Without wishing to be bound, it is believed that these compounds reduce the food intake of the host, thereby reducing the weight of the host.

Compounds of Formula I and II also exhibit anti-hyperglycemic activity. Thus, the compounds of the present invention are useful for treating diabetes. The compounds of the present invention exhibit anti-hypercholoesterolemic activity. Moreover, they lower triglycerides and are thus useful for treating hypertriglyceridemia. Inasmuch as high cholesterol and triglyceride levels are a primary cause for cardiovascular disease and other peripheral vascular diseases, the compounds of the present invention are useful for treating coronary heart disease and peripheral vascular disease.

Furthermore, the compounds of the present invention exhibit anti-glucocorticord activity. They are also useful for treating the adverse effects of enhanced glucocorticoid activity in a host. Thus, the compounds of Formula I and II are useful in treating, ameliorating, retarding and preventing the progression of unwanted conditions and/or symptoms or maladies relating to the enhanced level of glucocorticoids in the animal.

In addition, the compounds of Formula I and II herein are also effective in reducing the triglyceride concentration in patients who are insulin resistant. The term “insulin resistance” is defined as a disorder of glucose metabolism. More specifically, insulin resistance is the diminished ability of insulin to exert its biological action across a broad range of concentrations producing less than expected biologic effect. (see, e.g., Reaven, G. M., J. Basic & Clin. Phys. & Pharm. (1998) 9: 387-406 and Flier, J. Ann Rev. Med. (1983) 34:145-60). Insulin resistant persons have a diminished ability to properly metabolize glucose and respond poorly, if at all, to insulin therapy. Manifestations of insulin resistance include insufficient insulin activation of glucose uptake, oxidation and storage in muscle and inadequate insulin repression of lipolysis in adipose tissue and of glucose production and secretion in cells. Insulin resistance can cause or contribute to polycystic ovarian syndrome, Impaired Glucose Tolerance (IGT), gestational diabetes, hypertension, obesity, atherosclerosis and a variety of other disorders. Eventually, the insulin resistant individuals can progress to a point where a diabetic state is reached. The association of insulin resistance with glucose intolerance, an increase in plasma triglyceride and a decrease in high-density lipoprotein cholesterol concentrations, high blood pressure, hyperuricemia, smaller denser low-density lipoprotein particles, and higher circulating levels of plaminogen activator inhibitor-i), has been referred to as “Syndrome X” (see, e.g., Reaven, G. M., Physiol. Rev. (1995) 75: 473-486).

The compounds of Formula I and II are useful in modulating insulin resistance in a patient, e.g., mammal, the method comprising administering to the patient a therapeutically effective amount of the compound of Formula I or Formula II. Insulin resistance, may be a marker for generalized lipodystrophies. Thus, the compounds of the present invention are useful in treating lipodystrophies. Lypodystrophies have been known for over a century and are characterized by selective loss of body fat that can vary from small indentation or depressed areas in patients with localized lipodystrophies to near complete absence of adipose tissue in generalized lipodystrophies. More particularly, the compounds of Formula I and II are useful in treating generalized lipodystrophies.

Moreover, the compounds of Formula I and II are each useful for the treatment of Syndrome-X, also known as the insulin resistance syndrome. It includes hyperlipidemia, hyperinsulinemia, obesity, insulin resistance, insulin resistance leading to type-2 diabetes and diabetic complications thereof, i.e., diseases in which insulin resistance is the pathophysiological mechanismri .

Moreover, the compounds of Formula I and II are useful for treating hypertriglyceridemia, hypertension and coronary artery disease.

The compounds of Formula I and II are also useful in treating familial combined hyperlipidemia. Familial combined hyperlipidemia is a common disorder in which affected individuals have either hypercholesterolemia, hypercholesterolemia with hypertriglyceridemia or hypertriglyceridemia. These individuals are prone to premature atherosclerosis and coronary heart disease.

The compounds of the present invention have beneficial effects on the risk factors for the development of cardiovascular disease, type-2-diabetes, vascular disease and stroke. It is believed, without wishing to be bound, that elevated levels in the plasma of acute phase proteins and inflammatory cytokines, such as C-reactive proteins, interleukin-6, Pa, Al-1, or TNFα and the like are sensitive markers for systemic inflammation and for the development of cardiovascular disease, type-2-diabetes, vascular disease and stroke. Without wishing to be bound, it is believed that elevated amounts (relative to normal) are markers and/or present during the development of these diseases. By elevated amounts, it is meant that their concentrations in the plasma are greater than normal levels. For example, elevated levels of C-reactive proteins are present in the plasma in concentrations greater than 1.1 5 mg/l for both men and women. These amounts can be measured and determined using standard techniques known to one skilled in the art. Without wishing to be bound, it is believed that compounds of Formula I and II depress plasma levels of these, one or more of cytokines and acute phase proteins, such as C-reactive proteins, and the like, especially if given in effective doses, as defined herein. Thus, the treatment utilizing compounds of Formula I and II reduce the development or severity of cardiovascular disease and stroke.

The compounds of Formula I and II are also used to reduce the enhance glucocorticoid activity or actions in an animal, e.g. mammal.

Enhanced glucocorticoid action has been implicated as a cause for or as being associated with a number of ailments affecting animals, including mammals, especially man. For example, individuals may be immunosuppressed as a consequence of endogenous elevations in adrenal glucocorticoid (GCS) levels. These elevated levels can result from a variety of causes, including, but not limited to, stress and trauma (including, for example, post surgical trauma, and bum trauma), as a secondary consequence to any clinical condition which causes an elevated production of interleukin-1 (IL-1) or therapeutic treatment for a variety of clinical conditions. These elevated GCS levels can result in an imbalance in the production of essential interleukins. As a consequence thereof, the animals exhibit a depressed capacity to produce species of lymphokines which are essential to the development of protective forms of immunity. Plasma glucocorticoid steroid levels can also be elevated. exogenously as a consequence of therapeutic treatment for a variety of clinical conditions. In addition to the above, it is well known that certain essential functions to the immune system decline with age, a situation which correlates with elevations in adrenal output of glucocorticoid steroid and abatement in production of other types of adrenal steroid hormones.

Excess glucocorticoid actions is widely believed to be associated with mood changes, depression, vertigo, memory loss or impairment, disorientation, and the like.

Elevated glucocorticoid action are also linked with hippocampal pathology in aging rodents. Basal plasma corticosterone levels in aged rats have been found to correlate with hippocampal atrophy and spatial learning deficits. It has also been found that cumulative exposure to constant high levels of glucocorticoids disrupts electrophysiological function, leading to atrophy and ultimately the death of hippocampal neurons. It is widely believed that elevated glucocorticoid levels directly contribute to the development of cognitive impairments. Hippocampulatrophy has been reported in patients with Cushing's syndrome as a result of the hypersecretion of glucocorticoids.

Thus, the compounds of Formula I and Formula II have an anti-glucocorticoid effect. They are useful in treating, ameliorating, preventing or retarding the progression of the unwanted condition or symptom or malady in a patient relating to an enhanced glucocorticoid effect, said method comprising administering to said patient an anti-glucocrtiocoid effected amount of a compound Formula I or II.

An enhanced glucocorticoid activity, as defined herein, refers to an enhanced glucocorticoid effect relative to normal which is attributable or results from various factors, such as hypersecretion of the glucocorticoid, enhanced activity of 11-beta-hydroxysteriod dehydrogenase, which is an enzyme which converts cortisone to cortisol, the administration a glucocorticoid to patient, an enhanced concentration of giucocorticoid in the plasma relative to normal and the like. For example, the normal concentration of cortisol in the plasma in humans is about 7-20 ug/dL in the morning and about 3-13 ug/dL in the afternoon.

The compounds of the present invention are also useful in retarding immunosescence. Glucocorticoids, e.g., cortisol, are known to suppress the immune system and destroy lymphocytes in animals. The size of the thymus and the spleen are reduced in the presence of glucocorticoids, such as dexamethasone. The thymus and to some extent the spleen have a role in establishing the immunological capacity of the body. The thymus secretes hormones which are responsible for the production of cells with the capability of making antibodies and rejecting foreign bodies from the body. Moreover, both organs can produce lymphocytes and produce antibodies, which protect the body against invading microbes or foreign tissue. When the size of the thymus and spleen are reduced, their capacity to produce lymphocytes is also reduced, and the immune system is suppressed. Thus, as shown hereinabove, the compounds of the present protect against the atrophy of the spleen and thymus.

As one ages, the size of the spleen and the thymus also decreases. Further, as one ages, the cortisol levels also increase. Since glucocorticoids reduce the size of these two organs as one ages, the administration of compounds of Formula I and II retards the reduction of the size of these organs. Thus, the administration of the compounds of Formula I and II in anti-glucocorticoid effective amounts retards the suppression of the immune system through the aging process.

It is also known that cortisol and other glucocorticoids damage and/or cause the atrophy of the hypothalmus, and more specifically causes hippocampalatrophy. (See, Lupien, et al., Nature Neuroscience, 1998, Vol. 1, 69-73). It is believed that mental disorders and spatial performance are associated with hippocampal function. Sustained glucocorticoid exposure damages the hippocampus in humans. Elevated glucocorticoid levels have been linked to the damage of the hippocampus and the impairment of learning and memory. As indicated hereinabove, as one ages, the amount of cortisol in the body increases. This memory loss as one ages is believed to be attributable to the increase in the cortisol concentration in the body. Thus, the administration of compounds I and II in anti-glucocorticoid effective amounts retards the loss of memory.

The compounds of Formula I and II in therapeutically effective amounts are useful to inhibit unwanted biological or cellular responses to glucocorticoid steroids, e.g., (1) glucocorticoid-induced immune suppression, (2) glucocorticoid-induced bone loss, or (3) modulation of glucocorticoid-induced gene transcription or expression, e.g., increased or decreased expression. The present invention includes administration of a therapeutically effective amount of the compound of Formula I or II to a subject having or being susceptible to developing a glucocorticoid-associated symptom or condition, wherein the condition or symptom is prevented, detectably ameliorated or its onset of progression is detectably delayed or slowed. Thus, the compounds of Formula I and II can be used to prevent or ameliorate, e.g., immune suppression, decreased immune cell proliferation or adverse neurological effects (e.g., mood changes, depression, memory loss or impairment, disorientation, headache, vertigo and the like) of glucocorticoid steroids.

An excess or unwanted level of glucocorticoid steroids (“GCS”) in a subject such as a mammal or a human can arise from natural causes, such as infections, cancer or injury, or such levels can arise from the use of GCS to treat various disease conditions or symptoms. Other causes of increased values of cortisol include: adrenal hyperplasia, adrenal adenoma, adrenal carcinoma, pituitary tumor, ectopic ACTH syndrome, pregnancy, prior exercise, prior tobacco smoking, emotional or physical stress, exogenous estrogens, chronic renal failure, hyperthyroidism, exogenous cortisone or hydrocortisone and the like.

The GCS that are associated with such conditions or symptoms can be natural or synthetic. GCS levels that are associated with or that cause an unwanted condition or symptom can arise from a natural disease or from the administration of a natural or synthetic glucocorticoid steroid to a subject such as a mammal, e.g., human.

Thus, compounds of Formula I and II can be used to treat or prevent diseases that are associated therewith.

Moreover, corticosteroids are used to treat the following disorders: Achilles tendon disorders, Addison's disease, ankylosing spondylitis, asthma, athletic injury, atopic dermatitis, bacterial meningitis, carcinoid tumor, chickenpox, chronic lymphocytic leukemia, congenital adrenal hyperplasia, COPD, Crohn's disease, croup, cystic fibrosis, discoid lupus erythematosus, focal segmental glomerulosclerosis, gout, hay fever, Henoch-Schonlein purpura, hypercalcemia, idiopathic hypereosinophilic syndrome, idiopathic thrombocytopenic purpura, infectious mononucleosis lichen planus, minimal change disease, multiple myeloma, multiple schlerosis, neutropenia, nummular dermatitis, pemphigus, polyarteritis nodosa, polymyositis, psoriasis, rapidly progressive glomerulonephritis, recurrent aphthous stomatitis, respiratory failure, rheumatoid arthritis, sarcoidosis, spinal cord injury, systemic lupus erythematosus, tendenitis, toxic epodermal necrolysis, transplantation, tuberculosis, typhoid fever, ulcerative colitis and furthermore, Cortisol is used to treat the following disorders: Addison's disease, Cushing's disease, ectopic ACTH syndrome, hyponatremia, liver disease, pediatric cardiopulmonary resuscitation. The compounds of Formula I or II thus can limit the unwanted side effects of corticosteroids, such as GCS, without eliminating all of their beneficial, e.g., anti-inflammatory, effects. Thus, in some embodiments, a therapeutic treatment using a compound of Formula I and II is coadministered with one or more GCS. The GCS are used in a number of clinical situations, e.g., in chemotherapy, to decrease the intensity or frequency of flares or episodes of inflammation or autoimmune reactions in conditions such as rheumatoid arthritis, osteoarthritis, ulcerative colitis, bronchial asthma, psoriasis or systemic lupus erythematosus. Other side effects include but are not limited to, asceptic necrosis host defense alterations and the like. The compounds of Formula I and II reduce the sideeffects associated with the glucocorticoid treatment of these indications such as endocrine disorders, including adrenal cortical insufficiency, congenital adrenal hyperplasia, nonsuppurative thyroiditis, hypercalcemia associated with cancer, rheumatic disorders, including psoriatic arthritis, rheumatoid arthritis, ankylosing spondylitis, bursitis, acute nonspecific tenosynovitis, acute gouty arthritis, post-traumatic opsteoarthritis, synovitis of osteoarthritis, epicondylitis, collagen diseases, including systemic lupus erythematosus, acute rheynmatic carditis, dermatologic diseases, including pemphigus, bullous dermatitis herpetiformnis, severe erythema multiforme, exfoliative dermatitis, mycoses fungoides, severe psoriasis, severe seborrheic dermatitis, allergic states, including allergic rhinitis, bronchial asthma, contact dermatitis, atopic dermatitis, serum sickness, drug hypersensitivity reactions, ophthalmic diseases, including allergic conjunctivitis, keratitis, allergic comeal marginal ulcers, herpes zoster ophthalmicus, iritis and iridocyclitis, chorioretinitis, anterior segment inflammation, diffuse posterior uveitis and chorioditis, optic neuritis, sympathetic ophthalmia, respiratory diseases, including symptomatic sarcoidosis, Loeffler's syndrome, Berylliosis, pulmonary tuberculosis, aspiration pneumonitis, hematologic disorders, including idopathic and secondary thrombocytopenic purpura, iacquired hemolytic anemia, erythroblastopenia, congenital hypoplastic anemia, neoplastic diseases, including leukemias and lymphomas, edematous states, gastrointestinal diseases, including ulcerative colitis, regional enteritis, cerebral edema, including brain tumor, craniotomy, head injury, aging, and the like.

Adverse reactions that would be ameliorated by compounds of Formula I or II either through direct action or through allowing a lower dose of glucocorticoid to be used, for example, include but are not limited to fluid and electrolyte disturbances, including sodium retention, fluid retention, congestive heart failure, potassium loss, hypokalemic alkalosis, hypertension, muskuloskeletal, including muscle weakness, steroid myopathy, loss of muscle mass, osteoporaosis, vertebral compression fractures, asceptic necrosis, pthologic fracture of long bones, tendon rupture, gastrointestinal, including peptic ulcer, perforation of small and/or large bowel, pancreatitis, abdominal distention, ulcerative esophagitis,dermatologic, including impaired wound healing, thin fragile skin, petechiae & ecchymoses, erythema, increased sweating, suppressed reactions to skin tests, allergic dermatitis, urticaria, angioneurotic edema,neurologic, including convulsions, intracranial pressure, vertigo, headache, psychic disturbances, endocrine, including menstrual irregularities, cushingoid state, suppression of growth n chilkdren, adrenocortical and or pituitary unresponsivenesss, decreased carbohydrate tolerance, manifestations of latent diabetes mellitus, increased requirements for insulin or oral hypoglycemic agents in diabetics, hirsutism, ophthalmic, including posterior subcapsular cataracts, increased intraocular pressure, glaucoma, exophthalmus, metabolic, including negative nitrogen balance, cadiovascular, including myocardial rupture, other, including hypersensitivity, thromboembolism, weight gain, increased appetite, nausea, malaise, hiccups, nightmares, hallucinations, immune deficiencies, and the like.

The compounds of Formula I and II are useful to counteract the adverse effects or toxicities of glucocorticoids without negating all of the desired therapeutic capacity of the glucocorticoids. This allows the continued use, or a modified dosage of the glucocorticoid, e.g., an increased dosage, without an intensification of the side effects or toxicities or a decreased glucocorticoid dosage. The side-effects or toxicities that can be treated, prevented, ameliorated or reduced include one or more of the following: bone loss, reduced bone growth, enhanced bone resorption, osteoporosis, immunosuppression, increased susceptibility to infection, mood or personality changes, depression, headache, vertigo, high blood pressure or hypertension, muscle weakness, fatigue, nausea, malaise, peptic ulcers, pancreatitis, thin or fragile skin, growth suppression in children or preadult subjects, thromboembolism, cataracts, and edema.

The compounds of the present invention are also useful in treating auto-immune diseases, such as lupus erythermatosus, Graves' disease, multiple sclerosis, rheumatoid arthritis, inflammatory bowel disease, Sogren's syndrome, and the like.

The compounds of the present invention have anti-inflammatory activity and are useful for treating chronic inflammatory diseases, e.g., eczema, psoriasis, and the like.

The compounds of Formula I and II are effective in the treatment and/or prevention of the diseases identified hereinabove when administered to the patient in therapeutically or prophylactically effective amounts, respectively.

The compounds of the present invention can also be administered in combination with other compounds. In another embodiment, the compounds of Formula I or II or combination thereof can be used in combination with a statin for treating or preventing any of the diseases, maladies conditions or disorders described herein. As defined herein, a statin is a HMG-CoA-reductase inhibitor that inhibits HMG-CoA reductase. Thus, the statins are compounds having action of lowering blood cholesterol levels by inhibiting 3-hydroxy-3-methylglutaryl CoA (HMG-CoA) reductase. They have the advantages of lowering LDL.

As used herein, the term “fluasterone” refers to 16α-fluoro-5-androsten-17-one.

Moreover, the plural denotes the singular and vice versa.

The following non-limiting examples further illustrate the present invention.

### EXAMPLE 1

### 7-Hydroxy-16α-fluoro-5-androsten-17-one

A. 7-keto-16α-fluoro-androsten-17-one 36.75 g (125 mmoles) of 16α-fluoro-5-androsten-17-one was dissolved in 1.5 liters of benzene. 150 g Celite, 187.5 g pyridinium dichromate and 51.25 ml 70% t-butyl hydroperoxide were added thereto, and stirred mechanically for 24 hours at room temperature. 1.2 liters of diethyl ether was added thereto, and the resulting solution was cooled in an ice bath for a few minutes for a precipitate to form. The precipitate was filtered off, washed twice with fresh diethyl-ether and the combined filtrates, taken to dryness, collected. The precipitate was washed with diethyl ether. The 16α-fluoro-5-androsten-7,17-dione was separated out from the filtrate residue by flash chromatography using a 2 liter column containing silica gel (ICN, 32-64 μm) as the absorbent and hexane: ethyl acetate in a ratio of 82:18 (v/v) as the solvent which was added at a rate of 50 mL/min. 2.5 1 of void volume was collected and thereafter fractions of 0.5 liters each were collected. After collecting 16 fractions, the chromatographic solvent was changed to a mixture of hexane; ethyl acetate at a ratio of 77:23 (v/v). Fractions 11-33 contained the desired product, which was crystallized from methanol. 14.0 g of the 16α-fluoro-5-androsten-7,17-dione (mp 231-233° C.) was collected.

B. 7-keto-16α-fluoro-5-androsten-17-cyanohydrin acetate 5.0 g of 16α-fluoro-5-androsten-7,17-dione as prepared in A, was dissolved in 10 mL methylene chloride. 25 ml of acetone cyanohydrin and 1 ml of triethylamine were added thereto and stirred for 30 minutes at room temperature. The solvents were evaporated off using a rotoevaporator and a semi-crystalline residue was obtained. A methylene chloride water mixture (containing 3 parts solution weight methylene chloride and one part water) was added thereto.

The organic layer was separated and collected. The IR of the residue confirmed the product as the C-17 cyanohydrin. The residue was dried with sodium sulfate and after removing the sodium sulfate, the CH₂Cl₂ was evaporated off. The semi-crystalline residue thus formed was treated with 25 mL of pyridine and a 25 mL of acetic anhydride and the reaction was stirred for 48 hours at room temperature. The resulting product was concentrated by evaporation and then a mixture of toluene and reagent alcohol (90% anhydrous ethanol) was added to the mixture, and the mixture was azeotropically distilled. The resulting product was the C-17 cyanohydrin acetate which was purified by subjecting it to flash chromatography using silica gel (ICN, 32-64 μm) as the adsorbent and 80:20(v/v) hexane: ethyl acetate as the solvent which was added at a rate of 25 ml/min. After collecting 1 liter of void volume, 0.2 L fractions were collected. Fractions 12-22 contained the desired product.

C. 7-hydroxy-16α-fluoro-5-androsten-17-one The product of B (2.15 g) was placed in 95 mL of CH₂Cl₂ and 40 mL methanol in an ice bath. Sodium borohydride (750 mg) was added thereto. The reaction mixture was stirred at 0° C. for 1 hour. 0.75 mL of acetic acid and 0.75 mL pyridine were added thereto and subjected to azeotropic distillation until a solid residue was obtained., The product was dissolved in a solution of 375 mL methanol and 3.75 g KOH and refluxed for 1 hour. Methylenechloride/water mixture in a weight ratio of 3:1 was added thereto and the organic layer was collected and dried with sodium sulfate.

The dried product was subjected to HPLC on a 1″×25 cm column in which the adsorbent was silica gel (Hypersil, 5 μm) and the solvent used was a hexane ethyl acetate gradient program (linear gradient 0-40% ethyl acetate). Two of the products collected were 16α-fluoro-7α-hydroxy-5-androsten-17-one [(mp 150-151°) and a negative specific rotation ([α]_{D}=−72.0) and IR band at 1000 cm⁻¹] and 16α-fluoro-7β-hydroxy-5-androsten-17-one (mp 119-120°), a positive specific rotation [α]=+57.0° and (IR band at 1000 cm⁻¹).

### EXAMPLE2

### 7α+7-HYDROXY-16α-FLUORO-5α-ANDROSTAN-17-ONE

17.0 g of 16α-fluoro-5-androsten-17-β-ol was reacted with 20 mL of dihydropyran in 500 mL methylene chloride containing a few drops of concentrated hydrochloric acid for 2 hours at room temperature. The resulting product was washed with 5% sodium bicarbonate to produce the 17β THP anomers of 16α-fluoro-5androsten-17-β-ol. The anomers were separated by flash chromatography using hexane ethyl acetate (97:3 (v/v)) as the eluent and silica gel (ICN, 32-64 μm) as the adsorbent; the 17βTHP derivatives were collected and dried. These β derivatives were dissolved separately in benzene and subjected to allylic oxidation with pyridinium dichromate and 70% t-butyl hydroperoxide acid/celite in accordance with the procedure of Example 1. Catalytic hydrogenation with 5% H₂/Pd for 1 hour at 40 psi in a Parr shaker afforded the 16α-fluoro-7-keto-17-OTHP-5α-androstane. The resulting product is reduced with sodium borohydride to produce both the 7α and 7h-hydroxy-16α-fluoro-17-THP-5α-androstanes. The resulting product was treated with aqueous acetic acid/THF at room temperature to afford the corresponding 7-acetoxy-17 hydroxy derivatives. The 7-acetoxy product was oxidized with Jones reagent to produce the corresponding 17-keto derivative which was then hydrolyzed with methanol aqueous hydrochloric acid to form a mixture of the 7α- and 7β-hydroxy derivatives of 16α-fluoro-5α-androstan-17-one. The two isomers were separated by HPLC on silica gel as described above.

### EXAMPLE 3

### 16α-fluoro-5α,7β-dihydroxy-androstan-17-one

16α-fluoro-5-androsten-7-hydroxy-17-one is oxidized with m-perchlorobenzoic acid in chloroform to form the 5,6 epoxide. The epoxide is reacted with HBr to afford the 5-α-OH 6β-bromide which is reacted with zinc in acetic acid to afford the above-identified product.

The following experiments, compare the efficacy of a representative example of the present invention, e.g., 7-hydroxy-16α-fluoro-5-androsten-17-one compounds with 16α-fluoro-5-androstan-17-one in inhibiting TPA stimulated [³H] thymidine incorporation in mouse epidermis

It has been found that 16α-fluoro-5-androsten-17-one inhibits TPA-induced epidermal hyperplasia in mice. Epidermal hypeplasia is determined by measuring the stimulation in [³H] thymidine incorporation as well as epidermal DNA content of a 2×2 cm² section of skin twenty hours after TPA application.

In prior experiments, 16α-fluoro-5-androsten-17-one was administered in escalating doses by the oral subcutaneous, intraperitoneal, and buccal routes of administration, and the effect on TPA-stimulated epidermal [³H] thymidine incorporation and epidermal DNA content was determined. All four routes of administration produced a U-shaped dose response for [³H] thymidine incorporation, i.e., a suppression followed by a stimulation as the dose of 16α-fluoro-5-androsten-17one increased. DNA content, however, remained suppressed as the 16α-fluoro-5androsten-17-one dose increased.

Without wishing to be bound, it is believed that the increase in [³H] thymidine incorporation at high doses of 16α-fluoro-5-androsten-17-one is an artifact resulting from an increase in intracellular specific activity of [³H] thymidine 5′-triphosphate ([³H] TTP), as a consequence of glucose-6-phosphate dehydrogenase (G6PDH) inhibition. 16α-fluoro-5-androsten-17-one very likely reduces the endogenous TTP pool size, leading to an increase in the specific activity of the [³H] TTP pool size.

The lowest effective dose of 16α-fluoro-5-androsten-17-one for each route of administration which suppresses TPA-stimulated epidermal [³H] thymidine incorporation and DNA content is as follows:

Oral, 200 mg/kg

Subcutaneous, 2.5 mg/kg

Intraperitoneal, 5 mg/kg

Buccal, 2.5-5 mg/kg

### EXAMPLE 4

### Effect of 7α-hydroxy-16α-flouro-5-androsten-17-one versus 16α-flouro-5-androsten-17-one on TPA-Induced DNA Synthesis in Mouse Epidermis

In this experiment, the effect of treating mice subcutaneously (s.c.) with suspensions of 7α-hydroxy-16α-flouro-5-androsten-17-one and suspensions of 16aflouro-5-androsten-17-one were investigated.

Variousl6α-flouro-5-androsten-17 one suspensions (Q) were prepared. The vehicle was 95% saline (0.9% NaCl) and 5% Emulphor. For the 5 mg/kg s.c. suspension, 25.3 mg was suspended in 10 mL of Emulphor-saline. The other suspensions were made by diluting the 5 mg/kg suspension. For the 2.5 mg/kg suspension, 1 mL of 5mg/kg s.c. suspension was diluted to a final volume of 2mL, for the 1 m/kg suspension 1 mL of the 5 mg/kg suspension was made to a total of 5 mL, while for the 0.125 mg/kg suspension, 0.5 mL of the 5 mg/kg suspension was made to a final volume of 20 mL. Magnetic stir bars were added to the suspensions which were kept on a magnetic stirrer.

7α-hydroxy-16α-flouro-5-androsten-17-one was prepared as described hereinabove. For the 5 mg/kg s.c. suspension, 16.3 mg was suspended in 6.5 mL of Emulphor-saline. The other suspensions were made by diluting the 5 mg/kg suspension. For the 2.5 mg/kg, 1 mL of 5 mg/kg suspension was diluted to a final volume of 2 mL, for the 1 mg/kg suspension 1 mL of the 5 mg/kg suspension was made to total 5 mL, while for the 0.125 mg/kg suspension, 0.5 mL of the 5 mg/kg suspension was made up to final volume of 20 mL. Magnetic stir bars were added to the suspensions and the suspensions were kept on a magnetic stirrer.

Female CD-1 mice were obtained from Charles River Laboratories, Wilmington, Mass. at 44-46 days of age. The mice were housed two to three per cage in plastic shoebox cages on Alphacel bedding with 12 hours of alternating light and dark in the CAF Animal Facility, 6^{th} floor, Pharmacy Building. The mice had ad libitum access-to Purina 5015 chow and acidified tap water (pH≦2.6). The mice were allowed to acclimate to the facility for one week prior to use in an experiment. Three days after obtaining the mice, the mice were shaved and weighed. Only those mice showing no hair regrowth were used in the experiment.

Four days after the mice were shaved, they were treated as follows:

| | |
|---|---|
| Control | The mice were treated s.c. with 0.05 mL of Emulphor- |
| | saline vehicle. One hour after treatment with vehicle, the |
| | mice were treated topically with 0.2 mL of acetone. |
| TPA | The mice were treated s.c. with 0.05 mL of Emulphor- |
| | saline vehicle. One hour after treatment with vehicle, the |
| | mice were treated topically with 2 μ of TPA in 0.2 mL of |
| | acetone. |
| Q | The mice were treated s.c. with 0.05 mL of a 16α-fluoro- |
| 0.125 mg/kg | 5-androsten-17-one suspension which approximated a |
| subcutaneous | dose of 0.125 mg/kg. One hour after treatment with 16α- |
| | fluoro-5-androsten-17-one, the mice were treated |
| | topically with 2 μg of TPA in 0.2 mL of acetone. |
| Q | The mice were treated s.c. with 0.05 mL of a 16α-fluoro- |
| 2.5 mg/kg | 5-androsten-17-one suspension which approximated a |
| subcutaneous | dose of 1 mg/kg. One hour after treatment with 16α- |
| | fluoro-5-androsten-17-one, the mice were treated |
| | topically with 2 μg of TPA in 0.2 mL of acetone. |
| Q | The mice were treated s.c. with 0.05 mL of a 16α-fluoro- |
| 5 mg/kg | androsten-17-one, suspension which approximated a dose |
| subcutaneous | of 5 mg/kg. One hour after treatment with 16α-fluoro-5- |
| | androsten-17-one, the mice were treated topically with 2 |
| | μg of TPA in 0.2 mL of acetone. |
| 7 α-hydroxy | The mice were treated s.c. (at the nape) with 0.05 mL of a |
| 0.125 mg/kg | suspension of 7α-hydroxy-16α-fluoro-5-androsten-17-one |
| subcutaneous | which was approximately a dose of 1 mg/kg. One hour |
| | after treatment with 7α-hydroxy-16α-fluoro-5-androsten- |
| | 17-one, the mice were treated topically with 2 μg of TPA |
| | in 0.2 mL of acetone. |
| 7 α-hydroxy | The mice were treated s.c. (at the nape) with 0.05 mL of a |
| 1 mg/kg | suspension of 7α-hydroxy-16α-fluoro-5-androsten-17-one |
| subcutaneous | which was approximately a dose of 2.5 mg/kg. One hour |
| | after treatment with 7α-hydroxy-16α-fluoro-5-androsten- |
| | 17-one, the mice were treated topically with 2 μg of TPA |
| | in 0.2 mL of acetone. |
| 7 α-hydroxy | The mice were treated s.c. (at the nape) with 0.05 mL of a |
| 2.5 mg/kg | suspension of 7α-hydroxy-16α-fluoro-5-androsten-17-one |
| subcutaneous | which was approximately a dose of 2.5 mg/kg. One hour |
| | after treatment with 7α-hydroxy-16α-fluoro-5-androsten- |
| | 17-one 2 μg of TPA in 0.2 mL of acetone. |
| 7 α-hydroxy | The mice were treated s.c. (at the nape) with 0.05 mL of a |
| 5 mg/kg | suspension of 7α-hydroxy-16α-fluoro-5-androsten-17-one |
| subcutaneous | which was approximately a dose of 5 mg/kg. One hour |
| | after treatment with 7α-hydroxy-16α-fluoro-5-androsten- |
| | 17-one 2 μg of TPA in 0.2 mL of acetone. |

The mice were sacrificed 20 hours after treatment by an overdose of CO₂. Twenty minutes prior to sacrifice, the mice were injected with 60 μCi of [³H]thymidine (Amersham Batch 297). The mice were treated with a depilatory to remove any residual hair. A 2×2 cm² piece of skin was excised, placed in ice water for 30 seconds, then in 55° C. water for 30 seconds, then ice water again for 30 seconds. The epidermis was scrapped off using a scalpel and the scrapings were placed into ice cold 0.4N TCA (trichloroacetic acid). The scrapings were homogenized using a Tekmar Tissumizer (80% power for 30 seconds). The homogenates were centrifuged for 20 minutes at 3,000×g. The precipitates were washed 3× with 0.2N TCA, and 2× with absolute ethanol. The DNA in each sample was hydrolyzed with 0.5N TCA for 30 minuts at 90°. The tubes were centrifuged for 20 minutes at 3000×g. A 0.2 mL aliquot of each hydrolysate was counted in a LKB Rackbeta scintillation counter using Scintiverse II BD as the counting medium. DNA content was determined by the Burton diphenylamine assay.

The results are as follows:

BODY WEIGHTS:

| | | Q | | | | 7α-hydroxy-16α-fluoro-5-androsten-17-one | | | |
|---|---|---|---|---|---|---|---|---|---|
| | | 0.125 | | | | 0.125 | | 2.5 | |
| | | mg/kg | 1 mg/kg | 2.5 mg/kg, | 5 mg/kg, | mg/kg | 1 mg/kg | mg/kg | 5 mg/kg, |
| Control | TPA | s.c. | s.c. | s.c. | s.c. | s.c. | s.c. | s.c. | s.c. |
| 24.9 | 25.8 | 25.1 | 25.4 | 25.2 | 25.0 | 24.9 | 25.2 | 25.5 | 25.6 |
| 26.8 | 26.2 | 25.4 | 25.0 | 25.5 | 25.5 | 25.3 | 25.6 | 25.7 | 25.2 |
| 25.2 | 26.8 | | | | | | | | |
| 25.6 ± 1.0* | 26.2 ± 0.5 | 25.3 ± 0.2 | 25.2 ± 0.3 | 25.4 ± 0.2 | 25.3 ± 0.4 | 25.1 ± 0.4 | 25.4 ± 0.3 | 25.6 ± 0.1 | 25.4 ± 0.3 |

*Average

| | | corrected | | |
|---|---|---|---|---|
| Group | cpm | cpm | μg DNA | cpm/μg DNA |
| Control | 1,612.0 | 1,587.0 | 7.1 | 223.5 |
| | 1,758.0 | 1,733.0 | 8.2 | 211.3 |
| | 1,405.0 | 1,380.0 | 5.7 | 242.1 |
| | | | | 225.6 ± 15.5 |
| TPA | 7,749.0 | 7,724.0 | 14.0 | 551.7 |
| | 10,143.0 | 10,118.0 | 16.3 | 620.7 |
| | 8,999.0 | 8,974.0 | 13.3 | 674.7 |
| | | | | 647.7 ± 38.2 |
| Q | | | | |
| 0.125 mg/kg, s.c. | 3,496.0 | 3,471.0 | 10.0 | 347.1 |
| | 4,901.0 | 4,876.0 | 9.1 | 535.8 |
| | | | | 441.5 ± 133.4 |
| 1 mg/kg, s.c. | 1,836.0 | 1,811.0 | 8.9 | 203.5 |
| | 2,775.1 | 2,750.1 | 10.2 | 269.6 |
| | | | | 236.4 ± 46.5 |
| 2.5 mg/kg | 1,276.0 | 1,251.0 | 6.9 | 181.3 |
| | 1,232.0 | 1,207.0 | 7.3 | 165.3 |
| | | | | 173.3 ± 11.3 |
| 5 mg/kg | 8,274.0 | 8,249.0 | 7.6 | 1,085.4 |
| | 10,043.0 | 10,018.0 | 8.9 | 1,125.6 |
| | | | | 1105.5 ± 28.4 |

7α-hydroxy-16α-fluoro-5-androsten-17-one

| | | | | |
|---|---|---|---|---|
| 0.125 mg/kg, s.c. | 6,419.0 | 6,394.0 | 16.8 | 380.6 |
| | 4,483.0 | 4,458.0 | 15.4 | 289.5 |
| | | | | 335.1 ± 64.4 |
| 1 mg/kg, s.c. | 3,046.0 | 3,021.0 | 14.1 | 214.3 |
| | 3,648.0 | 3,623.0 | 13.1 | 276.6 |
| | | | | 245.5 ± 44.1 |
| 2.5 mg/kg | 4,503.0 | 4,478.0 | 12.0 | 373.2 |
| | 3,723.0. | 3,698.0 | 11.3 | 327.3 |
| | | | | 350.2 ± 32.5 |
| 5 mg/kg | 4,391.0 | 4,366.0 | 11.9 | 366.9 |
| | 4,234.0 | 4,209.0 | 11.9 | 353.7 |
| | | | | 360.3 ± 9.3 |

16α-fluoro-5-androsten-17-one produced a nadir in [³H] thymidine incorporation at 2.5 mg/kg with an overstimulation at 5 mg/kg.

As shown herein, the 7α-hydroxy-16α-fluoro-5-androsten-17-one inhibited [³H] thymidine incorporation and epidermal DNA content over the dose range with no apparent nadir or over stimulation.

### EXAMPLE 5

### Effect of 70β-hydroxy-16α-fluoro-5-androsten-17-one versus 16α-flouro-5-androsten-17-one on TPA-Induced DNA Synthesis in Mouse Epidermis

Various suspensions were prepared of 16α-fluoro-5-androsten-17-one as follows. The vehicle was 95% saline (0.9% NaCl) and 5% Emulphor. For the 5 mg/kg s.c. suspension, 27.8 mg was suspended in 11.4 mL of Emulphor-saline. The other suspensions were made by diluting the 5 mg/kg suspension. For the 2.5 mg/kg, 2 mL of 5 mg/kg suspension was diluted to a final volume of 4 mL, for the 1 mg/kg suspension 1 mL of the 5 mg/kg suspension was made to a total of 5 mL. Magnetic stir bars were added to the 16α-fluoro-5-androsten-17-one and the suspensions were kept on a magnetic stirrer.

7β-hydroxy-16α-fluoro-5-androsten-17-one, was prepared as in Ex. 1. For the 5 mg/kg suspension, 30.8 mg was suspended in 12.3 mL of Emulphor-saline. For the 3.75 mg/kg suspension, 3 mL of the 5 mg/kg suspension was diluted to a final volume of 4 mL, for the 2.5 mg/kg suspension, 1 mL of 5 mg/kg suspension was diluted to a final volume of 2 mL, for the 1.75 mg/kg suspension 2 mL of the 5 mg/kg suspension was made to a total of 5.6 mL while for the 1 mg/kg suspension, 1 mL of the 5 mg/kg suspension was diluted to a final dilution of 5 mL. All dilutions were made with Emulphor-saline. Magnetic stir bars were added, and the 7β-hydroxy-16α-fluoro5-androsten suspensions were kept on a magnetic stirrer.

Female CD-1 mice were obtained from Charles River Laboratories, Wilington, MA at 44-46 days of age. The mice were housed two to three per cage in plastic shoebox-cages on Alp acel bedding with 12 hours of alternating light and dark in the CAF Animal Facility, 6^{th} floor, Pharmacy Building. The mice had ad libitum access to Purina 5015 chow and acidified tap water (pH≦2.6). The mice were allowed to acclimate to the facility for one week prior to use in an experiment. Six days after obtaining the mice, the mice were shaved. Two days after the shaving, the mice were treated as follows:

| | |
|---|---|
| Control | The mice were treated s.c. with 0.05 mL of Emulphor- |
| | saline vehicle. One hour after treatment with vehicle, the |
| | mice were treated topically with 0.2 mL of acetone. |
| TPA | The mice were treated s.c. with 0.05 mL of Emulphor- |
| | saline vehicle. One hour after treatment with vehicle, the |
| | mice were treated topically with 2 μ of TPA in 0.2 mL of |
| | acetone. |
| Q 1 mg/kg | The mice were treated s.c. with 0.05 mL of a 16α-fluoro- |
| subcutaneous | 5-androsten-17-one suspension which approximated a |
| | dose of 1 mg/kg. One hour after treatment with 16α- |
| | fluoro-5-androsten-17-one, the mice were treated |
| | topically with 2 μg of TPA in 0.2 mL of acetone. |
| Q | The mice were treated s.c. with 0.05 mL of a 16α-fluoro- |
| 2.5 mg/kg | 5-androsten-17-one suspension which approximated a |
| subcutaneous | dose of 2.5 mg/kg. One hour after treatment with 16α- |
| | fluoro-5-androsten-17-one, the mice were treated |
| | topically with 2 μg of TPA in 0.2 mL of acetone. |
| Q | The mice were treated s.c. with 0.05 mL of a 16α-fluoro- |
| 5 mg/kg | 5-androsten-17-one suspension which approximated a |
| subcutaneous | dose of 5 mg/kg, One hour after treatment with 16α- |
| | fluoro-5-androsten-17-one, the mice were treated |
| | topically with 2 μg of TPA in 0.2 mL of acetone. |
| 7 β-hydroxy16α-fluoro | The mice were treated s.c. (at the nape) with 0.05 |
| -5-androsten-17-one | mL of a suspension of 7β- hydroxy-16α-fluoro-5- |
| 1 mg/kg | androsten-17-one which was approximately a |
| subcutaneous | dose of 1 mg/kg. One hour after treatment |
| | with 7β-hydroxy-16α-fluoro-5-androsten-17-one, |
| | the mice were treated topically with 2 μg of TPA |
| | in 0.2 mL of acetone. |
| 7 β-hydroxyfluasterone | The mice were treated s.c. (at the nape) with 0.05 |
| 1.75 mg/kg | mL of a suspension of 7β-hydroxy-fluasterone |
| subcutaneous | which was approximately a dose of 1.75 mg/kg. |
| | One hour after treatment with 7β-hydroxy-16α- |
| | fluoro-5-androsten-17-one, the mice were treated |
| | topically with 2 μg of TPA in 0.2 mL of acetone. |
| 7 β-hydroxy16α- | The mice were treated s.c. (at the nape) with 0.05 |
| fluoro-5-androsten-17- | mL of a suspension of 7β-hydroxy-16α-fluoro-5- |
| one2.5 mg/kg | androsten-17-one which was approximately a dose |
| subcutaneous | of 2.5 mg/kg. One hour after treatment with 7β- |
| | hydroxy-16α-fluoro-5-androsten-17-one, the mice |
| | were treated topically with 2 μg of TPA in 0.2 mL |
| | of acetone. |
| 7β-hydroxy16α-fluoro- | The mice were treated s.c. (at the nape) with 0.05 |
| 5-androsten-17-one | mL of a suspension of 7β-hydroxy-16α-fluoro-5- |
| 3.75 mg/kg | androsten-17-one which was approximately a dose |
| subcutaneous | of 3.75 mg/kg. One hour after treatment with 7β- |
| | hydroxy-16α-fluoro-5-androsten-17-one, the mice |
| | were treated topically with 2 μg of TPA in 0.2 mL |
| | of acetone. |
| 7β-hydroxy16α-fluoro- | The mice were treated s.c. (at the nape) with 0.05 |
| 5-androsten-17-one | mL of a suspension of 7β-hydroxy-16α-fluoro-5- |
| 5 mg/kg | androsten-17-one which was approximately a dose |
| subcutaneous | of 5 mg/kg. One hour after treatment with 7β- |
| | hydroxy-16α-fluoro-5-androsten-17-one, the mice |
| | were treated topically with 2 μg of TPA in 0.2 mL |
| | of acetone. |

The mice were sacrificed 20 hours after treatment by an overdose of CO₂. Twenty minutes prior to sacrifice, the mice were injected with 60 μCi of [³H]thymidine (Amersham Batch 297). The mice were treated with a depilatory to remove any residual hair. A 2×2 cm² piece of skin was excised, placed in ice water for 30 seconds, then in 55° C. water for 30 seconds, then in ice water again for 30 seconds. The epidermis was scraped off using a scapel and the scrapings wee placed into ice cold 0.4N TCA. The scrapings were homogenized using a Tekmar Tissumizer (80% power for 30 seconds). The homogenates were centrifuged for 20 minutes at 3,000×g. The precipitates were washed 3× with 0.2N TCA, and 2× with absolute ethanol. The DNA in each sample was hydrolyzed with 0.5N TCA, and 2× with absolute ethanol. The DNA in each sample was hydrolyzed with 0.5N TCA for 30 minutes at 90°. The tubes were centrifuged for 20 minutes at 3000×g. A 0.2 mL aliquot of each hydrolysate was counted in a LKB Rackbeta scintillation counter using Scintiverse II BD as the counting medium. DNA content was determined by the Burton diphenylamine assay.

The results are as follows:

BODY WEIGHTS:

| | | Fluasterone | | | | 7β-hydroxy-fluasterone | | | | | |
|---|---|---|---|---|---|---|---|---|---|---|---|
| | | 0.125 | | 2.5 | | 0.125 | | 1.75 | 2.5 | 3.75 | |
| | | mg/kg | 1 mg/kg | mg/kg, | 5 mg/kg, | mg/kg | 1 mg/kg | mg/kg | mg/kg | mg/kg | 5 mg/kg |
| Control | TPA | s.c. | s.c. | s.c. | s.c | s.c. | s.c. | s.c. | s.c. | s.c. | s.c. |
| 26.9 | 25.8 | 24.6 | 25.0 | 24.8 | 24.6 | 24.9 | 25.2 | 25.1 | 25.5 | 24.4 | 25.4 |
| 26.8 | 25.8 | 25.0 | 25.2 | 25.0 | 24.1 | 25.5 | 25.0 | 25.1 | 25.2 | 25.4 | 25.0 |
| 25.2 | 26.8 | | | | | | | | | | |
| 26.3 ± 1.0* | 26.1 ± 0.6 | 24.8 ± 0.3 | 25.1 ± 0.1 | 24.9 ± 0.1 | 24.4 ± 0.4 | 25.2 ± 0.4 | 25.1 ± 0.1 | 25.1 ± 0.0 | 25.4 ± 0.2 | 24.9 ± 0.7 | 25.2 ± 0.3 |

*Average

| Group | cpm | corrected cpm | μg DNA | cpm/μg DNA |
|---|---|---|---|---|
| Control | 871.0 | 845.0 | 9.2 | 91.8 |
| | 815.0 | 789.0 | 7.6 | 103.8 |
| | 1,014.0 | 988.0 | 8.9 | 111.0 |
| | | | | 102.2 ± 9.7 |
| TPA | 4,532.0 | 4,506.0 | 14.3 | 315.1 |
| | 9,717.0 | 9,691.0 | 17.1 | 566.7 |
| | 6,792.0 | 6,766.0 | 16.4 | 412.6 |
| | | | | 431.8 ± 127.4 |
| Fluasterone | | | | |
| 1 mg/kg, s.c. | 2,717.0 | 2,691.0 | 14.5 | 185.6 |
| | 2,767.0 | 2,741.0 | 17.7 | 154.9 |
| | | | | 170.3 ± 21.7 |
| 2.5 mg/kg, s.c. | 522.0 | 496.0 | 6.8 | 72.9 |
| | 592.0 | 566.0 | 6.3 | 89.8 |
| | | | | 81.4 ± 12.0 |
| 5 mg/kg | 2,218.0 | 2,192.0 | 8.2 | 267.3 |
| | 1,803.0 | 1,777.0 | 7.1 | 250.3 |
| | | | | 258.8 ± 12.0 |

7β-hydroxy-16α-fluoro-5-androsten-17-one

| | | | | |
|---|---|---|---|---|
| 1 mg/kg, s.c. | 4,527.0 | 4,501.0 | 14.6 | 308.3 |
| | 3,256.0 | 3,230.0 | 12.5 | 258.4 |
| | | | | 283.4 ± 35.3 |
| 1.75 mg/kg, s.c. | 1,471.0 | 1,445.0 | 10.0 | 144.5 |
| | 1,560.0 | 1,534.0 | 9.1 | 168.6 |
| | | | | 156.6 ± 17.0 |
| 2.5 mg/kg | 3,840.0 | 3,814.0 | 9.6 | 397.3 |
| | 5,887.0 | 5,861.0 | 10.4 | 563.6 |
| | | | | 480.5 ± 117.6 |
| 3.75 mg/kg | 4,732.0 | 4,706.0 | 8.3 | 567.0 |
| | 2,611.0 | 2,585.0 | 6.4 | 403.9 |
| | | | | 485.4 ± 115.3 |
| 5 mg/kg | 6,158.0 | 6,132.0 | 8.8 | 696.8 |
| | 3,934.0 | 3,908.0 | 6.4 | 610.6 |
| | | | | 653.7 ± 61.0 |

16α-fluoro-5-androstene produced a nadir in [³H] thymidine incorporation at 2.5 mg/kg and an overstimulation at 5 mg/kg. The 7β-hydroxy-16α-fluoro-5-androsten17-one, on the contrary produced an overstimulation at 2.5 mg/kg, and the nadir in [³H] thymidine incorporation is between lmg/kg and 2.5 mg/kg. Thus, 7β-hydroxy-16afluoro-5-androsten-17-one is much more active than the 16α-fluoro-5-androsten-17one.

The above preferred embodiments and examples are given to illustrate the scope and spirit of the present invention. The embodiments and examples described herein will make apparent to those skilled in the art other embodiments and examples. These other embodiments and examples are within the contemplation of the present invention. Therefore, the present invention should be limited only by the appended claims.

What is claimed is:

1. A method for treating diabetes in a mammal afflicted therewith comprising administering to said mammal an anti-diabetic effective amount of a compound of the formula: 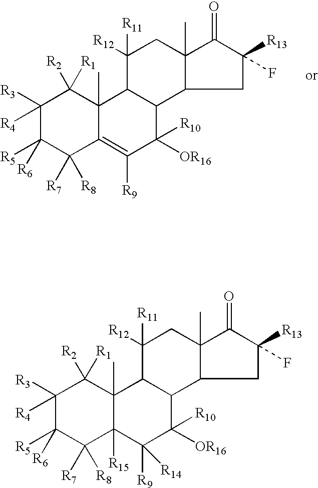
wherein
R₁, R₂, R₃, R₄, R₇, R₈, R₉, R₁₀, R₁₁, R₁₂, R₁₃, R₁₄ and R₁₆, are independently hydrogen or alkyl,
R₅ and R₆ are independently hydrogen or alkyl, and
R₁₅ is hydrogen, hydroxy or alkyl.

2. The method according to claim 1 wherein R₁₆ is hydrogen.

3. The method according to claim 1 wherein OR₁₆ is in the β-imposition.

4. The method according to claim 1 wherein R₅ and R₆ are hydrogen.

5. The method according to claim 1 wherein R₅ and R₆ are hydrogen and at most four of R₁, R₂, R₃, R₄, R₅, R₇, R₈, R₉, R₁₀, R₁₁, R₁₂, R₁₃ and R₁₆ are other than hydrogen.

6. The method according to claim 1 wherein R₁₅ is hydrogen, hydroxy or methoxy.

7. The method according to claim 1 in which the compound is administered buccally.

8. The method according to claim 1 wherein the compound has the formula: 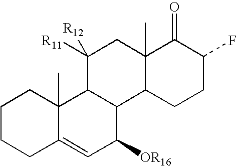
wherein R₁₁, R₁₂ and R₁₆ are independently hydrogen or lower alkyl.

9. The method according to claim 8 wherein R₁₁ and R₁₂ are hydrogen.

10. The method according to claim 8 wherein R₁₆ is hydrogen.

11. The method according to claim 9 wherein R₁₆ is hydrogen.

12. The method according to claim 8 wherein the compound is administered buccally.

13. The method according to claim 1 wherein the compound has the formula: 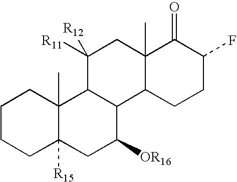
wherein R₁₁, R₁₂ and R₁₆ are independently hydrogen or lower alkyl and R₁₅ is hydrogen or hydroxy.

14. The method according to claim 13 wherein R₁₁ and R₁₂ are hydrogen.

15. The method according to claim 13 wherein R₁₅ is hydrogen or hydroxy.

16. The method according to claim 13 wherein R₁₅ is hydrogen or hydroxy, R₁₁ and R₁₂ are hydrogen and R₁₆ is hydrogen.

17. The method according to claim 13 wherein R₁₆ is hydrogen.

18. The method according to claim 13 wherein the compound is administered buccally.

19. The method for treating or preventing hypercholesterolemia in a mammal which method comprises administering to a mammal an anti-hypercholesterolemic effective amount of a compound of the formula 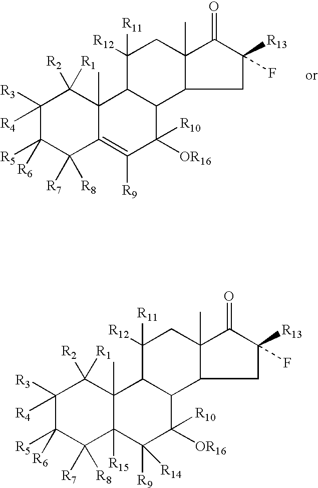
wherein
R₁, R₂, R₃, R₄, R₇, R₈, R₉, R₁₀, R₁₁, R₁₂, R₁₃, R₁₄ and R₁₆ are independently hydrogen or alkyl,
R₅ and R₆ are independently hydrogen or alkyl, and R₁₅ is hydrogen, hydroxy or alkyl.

20. A method of treating or preventing maladies or diseases resulting from a concentration of glucocorticoids in the plasma of a mammal that is greater than normal, said method comprising administering to said mammal, a therapeutic effective amount. of a compound of the formula: 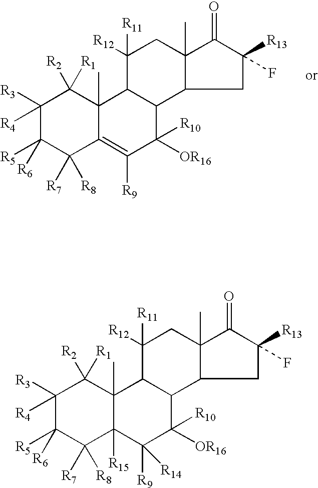
wherein
R₁, R₂, R₃, R₄, R₇, R₈, R₉, R₁₀, R₁₁, R₁₂, R₁₃, R₁₄ and R₁₆ are independently hydrogen or alkyl,
R₅ and R₆ are independently hydrogen or alkyl, and
R₁₅ is hydrogen, hydroxy or alkyl.

21. A method of reducing the glucocorticoid concentration in the plasma of a mammal comprising administering to said mammal a therapeutically effective amount of a compound of the formula: 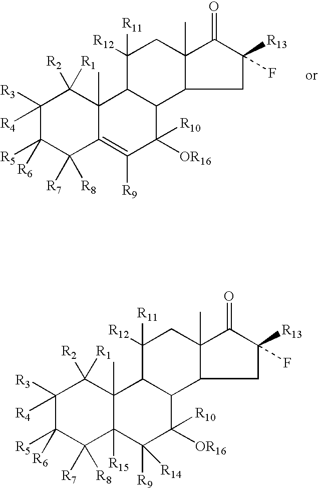
wherein
R₁, R₂, R₃, R₄, R₇, R₈, R₉, R₁₀, R₁₁, R₁₂, R₁₃, R₁₄ and R₁₆ are independently hydrogen or alkyl,
R₅ and R₆ are independently hydrogen or alkyl, and
R₁₅ is hydrogen, hydroxy or alkyl.

22. The method according to any one of claims 19-21 wherein R₁₆ is hydrogen.

23. The method according to claim 19, 20 or 21 wherein OR₁₆ is in the β-position.

24. The method according to claim 19, 20 or 21 wherein R₁₅ is hydrogen, hydroxy or methoxy.

25. The method according to claim 19, 20 or 21 wherein the compound is administered buccally.

26. The method according to claim 19, 20 or 21 wherein the compound has the formula: 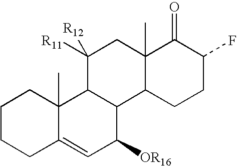
wherein R₁₁, R₁₂ and R₁₆ are independently hydrogen or lower alkyl.

27. The method according to claim 26 wherein R₁₁ and R₁₂ are hydrogen.

28. The method according to claim 26 wherein R₁₆ is hydrogen.

29. The method according to claim 26 wherein the compound is administered buccally.

30. The method according to claim 19, 20 or 21 wherein the compound is of the formula: 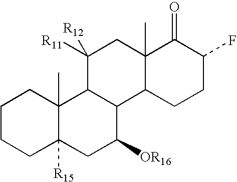
wherein R₁₁, R₁₂ and R₁₆ are independently hydrogen or lower alkyl and R₁₅ is hydrogen or hydroxy.

31. The method according to claim 30 wherein R₁₁ and R₁₂ are hydrogen.

32. The method according to claim 30 wherein R₁₆ is hydrogen.

33. The method according to claim 31 wherein R₁₆ is hydrogen.

34. The method according to claim 30 wherein R₁₅ is hydrogen or hydroxy.

35. The method according to claim 31 wherein R₁₅ is hydrogen or hydroxy.

36. The method according to claim 32 wherein R₁₅ is hydrogen or hydroxy.

37. The method according to claim 30 wherein R₁₅ is hydrogen or hydroxy, and R₁₁, R₁₂ and R₁₆ are hydrogen.

38. The method according to claim 30 wherein the compound is administered buccally.

39. The method according to any one of claims 1, 19, 20 or 21 wherein a statin is additionally present.

40. A compound of the formula: 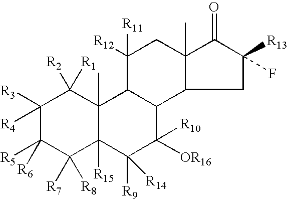
wherein
R₁, R₂, R₃, R₄, R₇, R₈, R₉, R₁₀, R₁₁, R₁₂, R₁₃, R₁₄ and R₁₆ are independently hydrogen or alkyl,
R₅ and R₆ are independently hydrogen or alkyl, and
R₁₅ is hydrogen, hydroxy or alkyl.

41. The compound according to claim 40 wherein R₁₆ is hydrogen.

42. The compound according to claim 40 wherein OR₁₆ is in the β-deposition.

43. The compound according to claim 40 wherein R₅ and R₆ are hydrogen.

44. The compound according to claim 40 wherein R₁₅ is hydrogen, hydroxy or methoxy.

45. The compound according to claim 40 wherein the compound has the formula: 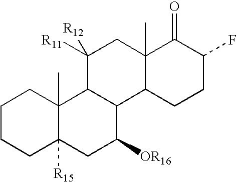
wherein R₁₁, R₁₂ and R₁₆ are independently hydrogen or lower alkyl and R₁₅ is hydrogen or hydroxy.

46. The compound according to claim 45 wherein R₁₁ and R₁₂ are hydrogen.

47. The compound according to claim 45 wherein R₁₅ is hydrogen or hydroxy.

48. The compound according to claim 46 wherein R₁₅ is hydrogen or hydroxy.

49. The compound according to claim 40 which is 16α-fluoro-7β-hydroxy-5α-androstan-17-one.

50. The compound according to claim 40 which is 16α-fluoro-7α-hydroxy-5α-androstan-17-one or 16α-fluoro-5α, 7β-dihydroxy-androstan-17-one, or 16α-fluoro-5α, 7α-dihydroxyandrostan-17-one.

51. The method according to any one of claims 1, 19, 20 or 21 wherein the compound is 16α-fluoro-7β-hydroxy-5-androsten-17-one.

52. The method according to any one of claims 1, 19, 20 or 21 wherein the compound is 16α-fluoro-7α-hydroxy-5-androsten-17-one, 16α-fluoro-7β-hydroxy-5α-androstan-17-one, 16α-fluoro-7α-hydroxy-5α-androstan-17-one, 16α-fluoro-5α,7α-dihydroxyandrostan-17-one or 16α-fluoro-5α, 7β-dihydroxyandrostan-17-one.

53. The compound according to claim 45 wherein R₁₆ is hydrogen.

54. The compound according to claim 46 wherein R₁₆ is hydrogen.

55. The compound according to claim 47 wherein R₁₆ is hydrogen.

56. The compound according to claim 48 wherein R₁₆ is hydrogen.
